(19) United States
(12) Patent Application Publication
(10) Pub. No.: US 2022/0275345 A1
(43) Pub. Date: Sep. 1, 2022

(54) METHODS FOR MAKING ORGANOID COMPOSITIONS

(71) Applicant: CHILDREN'S HOSPITAL MEDICAL CENTER, Cincinnati, OH (US)

(72) Inventors: Christopher Norman Mayhew, Hebron, KY (US); James Macormack Wells, Cincinnati, OH (US); Amy Lee Pitstick, Newport, KY (US)

(21) Appl. No.: 17/634,148

(22) PCT Filed: Aug. 11, 2020
(86) PCT No.: PCT/US2020/045809

§ 371 Date: Feb. 9, 2022

Related U.S. Application Data
(60) Provisional application No. 62/885,903, filed on Aug. 13, 2019.

(51) Int. Cl. C12N 5/071; C12M 3/00; C12M 1/32

(52) U.S. Cl. CPC C12N 5/0697; C12N 5/0671; C12N 5/0679; C12M 21/08; C12M 23/12; C12N 2506/45; C12N 2501/119; C12N 2501/115; C12N 2501/415; C12N 2501/11; C12N 2533/90; C12N 2513/00; C12N 2501/12

(57) ABSTRACT

Disclosed herein are organoids, or compositions thereof, produced through a process of aggregating gut endoderm monolayer and culturing the resultant gut endoderm aggregate. Examples of these aggregated organoids include but are not limited to aggregated liver organoids, aggregated gastric organoids, aggregated intestinal organoids, and aggregated colonic organoids.

### CROSS-REFERENCE TO RELATED APPLICATIONS

This application claims the benefit of priority of U.S. Provisional Patent Application No. 62/885,903, filed Aug. 13, 2019, which is hereby expressly incorporated by reference in its entirety.

### FIELD OF THE INVENTION

Aspects of the present disclosure relate generally to organoid compositions and methods of making the same involving the aggregation of precursor cells, for example, in formation plates.

### BACKGROUND

Human pluripotent stem cells (hPSCs; including both embryonic stem cells and induced pluripotent stem cells) represent a renewable resource for the generation of human 3-dimensional gastrointestinal tissue (e.g. human intestinal organoids; HIOs) that is organized into discrete epithelial and mesenchymal layers. For example, in HIOs, the epithelial contains all known intestinal epithelial cell types and exhibits several properties of functional intestine tissue including absorption, enteric hormone synthesis, and mucous secretion. Following transplantation into experimental animal models, HIOs undergo significant growth and maturation to resemble post-natal human intestine containing mucosa, submucosa, and muscularis propria. The epithelial cells arrange into crypt-villus structures containing adult stem cell activity/progenitor zones in crypts and mature epithelia capable of functions such as nutrient absorption and brush border enzyme activity. Consequently, organoids derived from pluripotent stem cells represent a physiologically-relevant and powerful tool to study intestinal development and disease, and also provide a novel platform for drug development. Furthermore, given that induced pluripotent stem cells can be derived from any individual, including those harboring intestinal disease, it is possible to generate disease/patient-specific organoids such as HIOs for personalized medicine applications. There is a lasting need for improved organoid compositions that more closely resemble in vivo tissue and methods of making the organoid compositions that are more robust, scalable, faster, and cost-effective.

### SUMMARY

Disclosed herein are methods of producing one or more aggregated organoids. In some embodiments, the methods comprise differentiating definitive endoderm to a gut endoderm monolayer and gut spheroids, separating the gut endoderm monolayer from the gut spheroids, dissociating the gut endoderm monolayer to a single cell suspension of gut endoderm cells, aggregating the single cell suspension of gut endoderm cells into one or more gut endoderm aggregates, and culturing the one or more gut endoderm aggregates to produce the one or more aggregated organoids. In some embodiments, the gut endoderm monolayer is adherent and the gut spheroids are detached and suspended in a growth medium. In some embodiments, the definitive endoderm has been differentiated from pluripotent stem cells. In some embodiments, the definitive endoderm has been differentiated from embryonic stem cells or induced pluripotent stem cells. In some embodiments, the definitive endoderm is human definitive endoderm. In some embodiments, the separating step comprises aspirating the growth medium and suspended gut spheroids from the gut endoderm monolayer. In some embodiments, the dissociating step comprises enzymatically dissociating the gut endoderm monolayer. In some embodiments, the gut endoderm monolayer is enzymatically dissociated with Accutase, Accumax, trypsin, trypsin/EDTA, collagenase, dispase, TrypLE Express, or TrypLE Select, or any combination thereof. In some embodiments, the aggregating step comprises aggregating the single cell suspension in hanging drops, centrifuging the single cell suspension in a “v” or “u”-bottomed microwell culture plate, aggregating the single cell suspension using an orbital shaker, or centrifuging the single cell suspension in a formation plate, or any combination thereof. In some embodiments, the formation plate is an Aggrewell plate. In some embodiments, each of the one or more gut endoderm aggregates comprises about 250, about 500, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, or about 10000 gut endoderm cells, or any number of gut endoderm cells within a range defined by any two of the aforementioned number of cells. In some embodiments, the culturing step comprises contacting the one or more gut endoderm aggregates with an extracellular matrix, or mimetic or derivative thereof. In some embodiments, the extracellular matrix, or mimetic or derivative thereof, comprises Matrigel.

In any of the embodiments disclosed herein, the gut endoderm monolayer is a foregut endoderm monolayer and the gut spheroids are foregut spheroids. In some embodiments, differentiating the definitive endoderm to the foregut endoderm monolayer and the foregut spheroids comprises contacting the definitive endoderm with one or more FGF signaling pathway activators, one or more Wnt signaling pathway activators, or one or more BMP signaling pathway inhibitors, or any combination thereof. In some embodiments, the one or more FGF signaling pathway activators comprise FGF4, the one or more Wnt signaling pathway activators comprise CHIR99021, or the one or more BMP signaling pathway inhibitors comprise Noggin, or any combination thereof. In some embodiments, the one or more aggregated organoids are aggregated liver organoids. In some embodiments, culturing the one or more gut endoderm aggregates to form the one or more aggregated liver organoids comprises contacting the one or more gut endoderm aggregates with one or more FGF signaling pathway activators, one or more BMP signaling pathway activators, retinoic acid, hepatocyte growth factor, dexamethasone, or Oncostatin M, or any combination thereof. In some embodiments, the one or more FGF signaling pathway activators comprise FGF2, or the one or more BMP signaling pathway activators comprise BMP4, or both. In some embodiments, the one or more aggregated organoids are an aggregated gastric organoids. In some embodiments, the one or more aggregated organoids are aggregated gastric organoids. In some embodiments, the one or more aggregated gastric organoids are aggregated antral gastric organoids. In some embodiments, culturing the one or more gut endoderm aggregates to form the one or more aggregated antral gastric organoids comprises contacting the one or more gut endoderm aggregates with EGF, retinoic acid, or one or more BMP signaling pathway inhibitors, or any combination thereof. In some embodiments, the one or more BMP signaling pathway inhibitors comprise Noggin

In any of the embodiments disclosed herein, the gut endoderm monolayer is a hindgut endoderm monolayer and the gut spheroids are hindgut spheroids. In some embodiments, differentiating the definitive endoderm to the hindgut endoderm monolayer and the hindgut spheroids comprises contacting the definitive endoderm with one or more FGF signaling pathway activators, or one or more Wnt signaling pathway activators, or both. In some embodiments, the one or more FGF signaling pathway activators comprise FGF4, or the one or more Wnt signaling pathway activators comprise CHIR99021, or both. In some embodiments, the one or more aggregated organoids are aggregated intestinal organoids. In some embodiments, culturing the one or more gut endoderm aggregates to form the one or more aggregated intestinal organoids comprises contacting the one or more gut endoderm aggregates with EGF, one or more Wnt signaling pathway activators, or one or more BMP signaling pathway inhibitors, or any combination thereof. In some embodiments, the one or more Wnt signaling pathway activators comprise R-spondin, or the one or more BMP signaling pathway inhibitors comprise Noggin, or both. In some embodiments, the one or more aggregated organoids are aggregated colonic organoids. In some embodiments, culturing the one or more gut endoderm aggregates to form the one or more aggregated colonic organoids comprises contacting the one or more gut endoderm aggregates with EGF, one or more Wnt signaling pathway activators, or one or more BMP signaling pathway activators, or any combination thereof. In some embodiments, the one or more Wnt signaling pathway activators comprise R-spondin, or the one or more BMP signaling pathway activators comprise BMP2, or any combination thereof.

In any of the embodiments disclosed herein, the one or more gut endoderm aggregates comprise at least 1, 10, 100, 200, 300, 400, 500, 600, 700, 800, 900, 1000, 2000, 3000, 4000, 5000, 6000, 7000, 8000, 9000, or 10000 gut endoderm aggregates, or any number of gut endoderm aggregates within a number defined by any two of the aforementioned number of gut endoderm aggregates. In some embodiments, each of the one or more gut endoderm aggregates comprise: a diameter that is within ±10%, ±90%, ±8%, ±70%, ±6%, ±5%, ±4%, ±3%, ±2%, or ±1% from the average diameter of the one or more gut endoderm aggregates, or any diameter within a range defined by any two of the aforementioned diameters; or a volume that is within ±10%, ±9%, ±8%, ±70%, ±6%, ±5%, ±4%, ±3%, ±2%, or ±1% from the average volume of the one or more gut endoderm aggregates, or any volume within a range defined by any two of the aforementioned volumes; or both.

In any of the embodiments disclosed herein, the methods further comprise transplanting the one or more aggregated organoids to a recipient subject. In some embodiments, the recipient subject is a mammal. In some embodiments, the recipient subject is a human.

Also disclosed herein are any of the one or more aggregated organoid produced by any one of the methods disclosed herein. Also disclosed herein is a plurality of gut endoderm aggregates. In some embodiments, the plurality of gut endoderm aggregates comprise at least 1000, 2000, 3000, 4000, 5000, 6000, 7000, 8000, 9000, or 10000 gut endoderm aggregates, or any number of gut endoderm aggregates within a number defined by any two of the aforementioned number of gut endoderm aggregates; wherein each of the plurality of gut endoderm aggregates comprises: a diameter that is within ±10%, ±90, 8%, ±7%, ±6%, ±5%, ±4%, ±3%, ±2%, or ±1% from the average diameter of the plurality of gut endoderm aggregates, or any diameter within a range defined by any two of the aforementioned diameters; or a volume that is within ±10%, ±9%, ±8%, ±7%, ±6%, ±5%, ±4%, ±3%, ±2%, or ±1% from the average volume of the plurality of gut endoderm aggregates, or any volume within a range defined by any two of the aforementioned volumes; or both. In some embodiments, the plurality of gut endoderm aggregates are derived from the same subject. Also disclosed herein is a formation plate. In some embodiments, the formation plate comprises a plurality of microwells and the plurality of gut endoderm aggregates of claim 38 or 39, wherein each of the plurality of microwells comprises a single gut endoderm aggregate of the plurality of gut endoderm aggregates. In some embodiments, for any one of the plurality of gut endoderm aggregates disclosed herein, or any one of the formation plates disclosed herein, the plurality of gut endoderm aggregates is produced according to any one of the methods disclosed herein.

Embodiments of the present disclosure provided herein are described by way of the following numbered alternatives:

1. A method for making an organoid composition comprising:

dissociating a hindgut endoderm (HGE) to form an HGE-derived single cell population;

aggregating said HGE-derived single cell population in a formation plate;

culturing said HGE-derived single cell population in said formation plate to form an aggregate; and

culturing said aggregate with EGF, a BMP signaling pathway activator, and a Wnt signaling pathway activator until an intestinal organoid is formed.

2. The method of alternative 1, wherein said hindgut endoderm (HGE) is obtained from definitive endoderm (DE).

3. The method of alternative 2, wherein said DE is cultured with an FGF signaling pathway activator and a Wnt signaling pathway activator to form said hindgut endoderm (HGE).

4. The method of any preceding alternative wherein said organoid is obtained from a precursor cell.

5. The method of any preceding alternative wherein said precursor cell is an induced pluripotent stem cell.

6. The method of alternative 1, wherein said HGE-derived single cell population comprises hindgut endoderm cells.

7. The method of any preceding alternative wherein said aggregate comprises about 1000 hindgut endoderm cells, or about 2000 hindgut endoderm cells, or about 3000 hindgut endoderm cells, or about 4000 hindgut endoderm cells, or about 5000 hindgut endoderm cells.

8. The method of any preceding alternative wherein said aggregate is contacted with anti-Adherence Rinsing Solution.

9. The method of any preceding alternative, comprising contacting said aggregate with a 3D structure, preferably Matrigel (basement membrane matrix), and further culturing said aggregate until an organoid is formed.

10. The method of any preceding alternative, wherein said formation plate is selected from a microwell culture plate, a V bottom microwell culture plate, a hanging drop culture plate, or a plate capable of physically aggregating a cell population.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1 depicts an embodiment of a timeline of hPSC to HIO differentiation and development.

FIGS. 2A-C depict embodiments of a preparation of a gut endoderm monolayer for single cell dissociation and aggregation.

FIGS. 3A-B depict embodiments of a formation plate and aggregation of cells to form aggregates.

FIGS. 4-6 depict embodiments of a formation plate or components thereof.

FIG. 7A depicts an embodiment of a schematic of existing spheroid production protocol for human intestinal organoid (HIO) generation.

FIG. 7B depicts an embodiment of inter-experimental variability of spheroid production using embodiments of existing protocols.

FIG. 7C depicts an embodiment of inter-line variability of spheroid production using embodiments of existing protocols.

FIG. 7D depicts an embodiment of exemplary images corresponding to FIG. 7C.

FIG. 8 depicts an embodiment of uniformly CDX2+ hindgut endoderm produced by embodiments of existing protocols.

FIG. 9A depicts an embodiment of a schematic of aggregation-based spheroid production protocols for HIO generation.

FIG. 9B depicts an embodiment of successful, uniform aggregation of multiple hPSC lines.

FIG. 9C depicts an embodiment of images depicting significantly increased yield of uniform spheroids producing by an aggregation method.

FIG. 9D depicts an embodiment of significantly increased yield of spheroids per well from HGE aggregation.

FIG. 9E depicts an embodiment of different organization of epithelial and mesenchymal cells in detached spontaneous spheroids and aggregated spheroids.

FIG. 10A depicts an embodiment of images showing that detached spontaneous spheroids and aggregated spheroids are morphologically indistinguishable after growth in Matrigel.

FIG. 10B depicts an embodiment of images showing indistinguishable organization of epithelial and mesenchymal cells in detached spontaneous spheroids and aggregated spheroids 3 days after embedding in Matrigel.

FIG. 11A depicts an embodiment of images showing that growth and morphology of aggHIOs is indistinguishable from spontaneous HIOs.

FIG. 11B depicts an embodiment of images showing that AggHIOs comprise CDX2+ intestinal epithelial and Emilin1+ mesenchyme.

FIG. 11C depicts an embodiment of images showing that both spontaneous detached HIOs and AggHIOs are patterned to proximal small intestine.

FIG. 12A depicts an embodiment of images showing that AggHIOs undergo robust growth and maturation following in vivo transplantation.

FIG. 12B depicts an embodiment of immunofluorescence analysis of mature small intestinal markers.

FIG. 13A depicts an embodiment of a schematic for the generation of antral human gastric organoids (aHGOs) by aggregation.

FIG. 13B depicts an embodiment of images showing that aHGOs derived from aggregated foregut endoderm are morphologically indistinguishable from spontaneous aHGOs.

FIG. 13C depicts an embodiment of images showing that expression of gastric epithelial markers is indistinguishable between spontaneous and aggregated aHGOs.

FIG. 14A depicts an embodiment of a schematic for the generation of human colonic organoids (HCOs) by aggregation.

FIG. 14B depicts an embodiment of images showing that HCOs derived from aggregated hindgut endoderm spheroids are morphologically indistinguishable from HCOs derived from spontaneous spheroids.

FIG. 14C depicts an embodiment of images showing that expression of the colonic epithelial marker SATB2 is indistinguishable between HCOs derived from spontaneous or aggregated hindgut endoderm.

FIG. 15 depicts an embodiment of a schematic for the generation of human liver organoids (HLOs) by aggregation.

FIG. 16A depicts an embodiment of images showing population density of mesoderm (as detected by T expression), and definitive endoderm (as detected by FOXA2 expression) in definitive endoderm cultures differentiated from PSCs.

FIG. 16B depicts an embodiment of the quantification of mesoderm and definitive endoderm population percentages in the culture of FIG. 16A.

FIG. 16C depicts an embodiment of images showing population density of mesenchyme (as detected by FOXF1 expression), and gut endoderm (as detected by FOXA2 expression) in foregut and hindgut endoderm monolayer cultures differentiated from PSC-derived definitive endoderm.

FIG. 16D depicts an embodiment of the quantification of mesenchyme and endoderm population percentages in the culture of FIG. 16C.

FIG. 16E depicts an embodiment of the quantification of proliferating mesoderm and proliferating endoderm comparing day 3 definitive endoderm and day 7 hindgut endoderm cultures.

### DETAILED DESCRIPTION

Current technologies for organoid generation rely on the stepwise in vitro differentiation of hPSCs to organ tissue lineages. For example, for gastrointestinal organoids, the stem cells are differentiated first to definitive endoderm (DE) cells and subsequently foregut endoderm (FGE) or hindgut endoderm (HGE) intermediates (about 7 days of culture) during which spontaneous morphogenesis occurs and results in the formation and detachment of 3D spheroids that resemble the embryonic gut tube. These spheroids are them embedded in an extracellular matrix, or a mimetic or derivative thereof (e.g. Matrigel), and cultured in media that promotes growth and organ differentiation. After about 28 days in these conditions (day 35 of culture total), human organoids can be harvested and be used for purposes such as studying organ function and morphology, drug screening, or engraftment to animal models for further growth and maturation.

However, several limitations exist for current organoid generation methods. In particular, there is significant variability in the efficiency of spontaneous spheroid production and detachment between different hPSC lines. Even with lines known to have robust ability to produce spontaneous spheroids, there is significant experiment-to-experiment variability in spontaneous spheroid production and detachment. There is also significant variability in the efficiency of spontaneous spheroid production and detachment from well-to-well within a specific organoid generation experiment. Spontaneous spheroid formation often occurs in large “chains” of multiple attached spheroids. The size of spontaneous spheroids generated from line-to-line can vary significantly. Therefore, the reliance on spontaneous morphogenesis is associated with inefficient and inconsistent spheroid generation. Furthermore, these methods are not well suited for the increased scalability required for biopharmaceutical manufacturing applications.

Provided herein are improved methods of making organoids, or compositions thereof, that overcomes one or more limitations of existing methods. In some embodiments, the disclosed methods eliminate or reduce the low efficiency of spontaneous spheroid production associated with line-to-line, experiment-to-experiment, and well-to-well variability, and further provide methods for improved scalability.

The methods disclosed herein takes advantage of the ability of hPSC-derived cells to self-organize upon aggregation. For gastrointestinal organoids, hPSCs are differentiated to DE and then FGE or HGE using standard methods and cells that remain attached to the cell culture plate, including in instances where no spontaneous morphogenesis and spheroid formation and detachment are detected, are then subjected to dissociated to single cells. In some embodiments, single FGE or HGE cells are then aggregated in a formation plate, for example overnight. In some embodiments, the formation plate is an Aggrewell plate (StemCell Technologies). In some embodiments, aggregates are then harvested and embedded in Matrigel for growth and differentiation to organoids (e.g. intestinal organoids). Analysis of HIOs derived via the improved aggregation method demonstrates no significant difference compared to HIOs derived via spontaneous spheroid generation in the same experiment. Furthermore, HIOs derived from aggregated HGE retain the ability to undergo growth to mature human intestinal tissue following engraftment to a mouse model. In some embodiments, processes for differentiating other types of organoids known in the art may be employed with the methods described herein.

Disclosed herein are aggregated organoids, and compositions thereof, and methods of making the same involving the aggregation of a single cell suspension of a gut endoderm monolayer (as opposed to gut spheroids) and subsequent culturing of the aggregate to form aggregate organoids. The methods disclosed herein produce organoids with greater reliability and reproducibility than traditional methods known in the art, and has implications in the feasibility of scaling-up organoid manufacturing. These aggregated organoids can be used for purposes such as drug screening or personalized medicine and are suitable for transplantation, for example, autologously or allogeneically to a subject, such as a human or other mammal, or xenogeneically into immunocompromised animals. In some embodiments, the aggregate organoids are liver, gastric, antral gastric, fundic gastric, intestinal, or colonic organoids. In some embodiments, the aggregate organoids are derived from cells isolated from a patient. Methods of producing organoids can be found in U.S. Pat. Nos. 9,719,068 and 10,174,289, and PCT Publications WO 2016/061464, WO 2017/192997, WO 2018/106628, WO 2018/200481, WO 2018/085615, WO 2018/085622, WO 2018/085623, WO 2018/226267, WO 2020/023245, each of which is hereby expressly incorporated by reference in its entirety.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

For clarity of disclosure, to the extent that spatial terms such as “upper,” “lower,” “longitudinal,” “lateral,” “transverse,” “inward,” “outward,” or the like are used herein with reference to the drawings, it will be appreciated that such terms are used for exemplary description purposes only and are not intended to be limiting or absolute. In that regard, it will be understood that instruments such as those disclosed herein may be used in a variety of orientations and positions not limited to those shown and described herein.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present disclosure include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present disclosure include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

The term “extracellular matrix” as used herein has its plain and ordinary meaning in light of the specification and refers to any biological or synthetic compound, substance, or composition that enhances cell attachment and/or growth. Any extracellular matrix, as well as any mimetic or derivative thereof, known in the art can be used for the methods disclosed herein. Some examples of extracellular matrices, or mimetics or derivative thereof, include but are not limited to cell-based feeder layers, polymers, proteins, polypeptides, nucleic acids, sugars, lipids, poly-lysine, poly-ornithine, collagen, gelatin, fibronectin, vitronectin, laminin, elastin, tenascin, heparan sulfate, entactin, nidogen, osteopontin, basement membrane, Matrigel, hydrogel, PEI, WGA, or hyaluronic acid, or any combination thereof.

Some embodiments described herein relate to pharmaceutical compositions that comprise, consist essentially of, or consist of an effective amount of a cell composition described herein and a pharmaceutically acceptable carrier, excipient, or combination thereof. A pharmaceutical composition described herein is suitable for human and/or veterinary applications.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The disclosure herein generally uses affirmative language to describe the numerous embodiments. The disclosure also includes embodiments in which subject matter is excluded, in full or in part, such as substances or materials, method steps and conditions, protocols, or procedures.

### Differentiation of PSCs

In some embodiments, PSCs, such as ESCs and iPSCs, undergo directed differentiation in a stepwise manner first into definitive endoderm (DE) cells, then into foregut or hindgut lineages, and into gastrointestinal tissue. In some non-limiting embodiments, the PSCs may comprise H1 hESCs, iPSC72_3, iPSC75_1, or iPSC285_1, or any combination thereof. In some embodiments, PSCs undergo directed differentiation in a non-stepwise manner where molecules (e.g. growth factors, ligands) for promoting DE formation and for subsequent tissue formation are added at the same time. In some embodiments, directed differentiation is achieved by selectively activating certain signaling pathways in the iPSCs and/or DE cells. In some embodiments, the signaling pathways include but not limited to the Wnt signaling pathway; Wnt/APC signaling pathway; FGF signaling pathway; TGF-beta signaling pathway; BMP signaling pathway; Notch signaling pathway; Hedgehog signaling pathway; LKB signaling pathway; and Par polarity signaling pathway. Each of the listed signaling pathways have signaling pathway activators and signaling pathway inhibitors which are conventionally known in the art.

The definitive endoderm gives rise to the gut tube. The anterior DE forms the foregut and its associated organs including the esophagus, lungs, stomach, liver and pancreas and the posterior DE forms the midgut and hindgut, which forms the small and large intestines and parts of the genitourinary system. Studies using mouse, chick and frog embryos suggest that establishing the anterior-posterior pattern in DE at the gastrula stage is a prerequisite for subsequent foregut and hindgut development. The Wnt and FGF signaling pathways are critical for promoting either posterior endoderm/hindgut or anterior endoderm/foregut fate. In hindgut, the simple cuboidal epithelium first develops into a pseudostratified columnar epithelium, then into villi containing a polarized columnar epithelium and a proliferative zone at the base of the villi, which corresponds with the presumptive progenitor domain.

Any methods for producing definitive endoderm from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some embodiments, human embryonic stem cells are used to produce definitive endoderm. In some embodiments, human embryonic germ cells are used to produce definitive endoderm. In some embodiments, iPSCs are used to produce definitive endoderm. In some embodiments, human iPSCs (hiPSCs) are used to produce definitive endoderm. In some embodiments, PSCs are first modified before differentiating into definitive endoderm. In some embodiments, the PSCs are genetically modified, such as to express an exogenous nucleic acid or protein, before differentiating into definitive endoderm.

In some embodiments, the embryonic stem cells or germ cells or iPSCs are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the embryonic stem cells or germ cells or iPSCs are treated with one or more small molecule compounds, activators, inhibitors, or growth factors at a concentration that is, is about, is at least, is at least about, is not more than, or is not more than about, 10 ng/mL, 20 ng/mL, 50 ng/mL, 75 ng/mL, 100 ng/mL, 120 ng/mL, 150 ng/mL, 200 ng/mL, 500 ng/mL, 1000 ng/mL, 1200 ng/mL, 1500 ng/mL, 2000 ng/mL, 5000 ng/mL, 7000 ng/mL, 10000 ng/mL, or 15000 ng/mL, or any concentration that is within a range defined by any two of the aforementioned concentrations, for example, 10 ng/mL to 15000 ng/mL, 100 ng/mL to 5000 ng/mL, 500 ng/mL to 2000 ng/mL, 10 ng/mL to 2000 ng/mL, or 1000 ng/mL to 15000 ng/mL. In some embodiments, concentration of the one or more small molecule compounds, activators, inhibitors, or growth factors is maintained at a constant level throughout the treatment. In some embodiments, concentration of the one or more small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the ESCs, germ cells, or iPSCs are cultured in growth media that supports the growth of stem cells. In some embodiments, the ESCs, germ cells, or iPSCs are cultured in stem cell growth media. In some embodiments, the stem cell growth media is RPMI 1640, DMEM, DMEM/F12, mTeSR 1, mTeSR Plus, DE Differentiation, Hindgut Endoderm Differentiation, Gut Base, or Complete Sato media. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10⁰/a, 11%, 12%, 13%, 14%, 15%, 16%, 17%, 18%, 19%, or 20%, or any percentage within a range defined by any two of the aforementioned concentrations, for example 0% to 200%, 0.2% to 100%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, populations of cells enriched in definitive endoderm cells are used. In some embodiments, the definitive endoderm cells are isolated or substantially purified. In some embodiments, the isolated or substantially purified definitive endoderm cells express one or more (e.g. at least 1, 3) of SOX17, FOXA2, or CXRC4 markers to a greater extent than one or more (e.g. at least 1, 3, 5) of OCT4, AFP, TM, SPARC, or SOX7 markers.

In some embodiments, definitive endoderm cells and hESCs are treated with one or more growth factors. Such growth factors can include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, a Wnt protein or combinations of any of these growth factors. For example, in human, Wnt proteins include but are not limited to Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16.

In some embodiments, activin-induced definitive endoderm (DE) can further undergo FGF and/or Wnt induced anterior or posterior endoderm pattering, foregut or hindgut specification and morphogenesis, and finally gastrointestinal growth, morphogenesis and cytodifferentiation into functional gastrointestinal cell types. In some embodiments, PSCs are efficiently directed to differentiate in vitro into gastrointestinal epithelium or mesenchyme that includes secretory, endocrine and absorptive cell types. It will be understood that molecules such as growth factors can be added to any stage of the development to promote a particular type of gastrointestinal tissue formation.

Human gastrointestinal development in vitro occurs in stages that approximate fetal gut development; endoderm formation, anterior or posterior endoderm patterning, foregut or hindgut morphogenesis, fetal gut development, epithelial morphogenesis, formation of a presumptive progenitor domain, and differentiation into functional cell types.

It will be understood by one of skill in the art that altering the concentration, expression or function of one or more Wnt signaling proteins in combination with altering the concentration, expression, or function of one or more FGF proteins can give rise to directed differentiation in accordance of the present disclosure. In some embodiments, cellular constituents associated with the Wnt and/or FGF signaling pathways, for example, natural inhibitors, antagonists, activators, or agonists of the pathways can be used to result in inhibition or activation of the Wnt and/or FGF signaling pathways. In some embodiments, siRNA and/or shRNA targeting cellular constituents associated with the Wnt and/or FGF signaling pathways are used to inhibit or activate these pathways.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. FGFs are key players in the processes of proliferation and differentiation of wide variety of cells and tissues. In humans, 22 members of the FGF family have been identified, all of which are structurally related signaling molecules. Members FGF1 through FGF10 all bind fibroblast growth factor receptors (FGFRs). FGF1 is also known as acidic fibroblast growth factor, and FGF2 is also known as basic fibroblast growth factor (bFGF). Members FGF11, FGF12, FGF13, and FGF14, also known as FGF homologous factors 1-4 (FHF1-FHF4), have been shown to have distinct functional differences compared to the FGFs. Although these factors possess remarkably similar sequence homology, they do not bind FGFRs and are involved in intracellular processes unrelated to the FGFs. This group is also known as “iFGF.” Members FGF15 through FGF23 are newer and not as well characterized. FGF15 is the mouse ortholog of human FGF19 (hence there is no human FGF15). Human FGF20 was identified based on its homology to Xenopus FGF-20 (XFGF-20). In contrast to the local activity of the other FGFs, FGF15/FGF19, FGF21 and FGF23 have more systemic effects.

In some embodiments, it will be understood by one of skill in the art that any of the FGFs can be used in conjunction with a protein from the Wnt signaling pathway. In some embodiments, the FGF used is one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23.

Differentiation of PSCs into DE culture and subsequently into various intermediate mature gastrointestinal cell types can be determined by the presence of stage-specific cell markers. In some embodiments, expression of representative cellular constituents is used to determine DE formation. The representative cellular constituents include but are not limited to CMKOR1, CXCR4, GPR37, RTN4RL1, SLC5A9, SLC40A1, TRPA1, AGPAT3, APOA2, C20orf56, C21orf129, CALCR, CCL2, CER1, CMKOR1, CRIP1, CXCR4, CXorf1, DIO3, DIO30S, EB-1, EHHADH, ELOVL2, EPSTI1, FGF17, FLJ10970, FLJ21195, FLJ22471, FLJ23514, FOXA2, FOXQ1, GATA4, GPR37, GSC, LOC283537, MYL7, NPPB, NTN4, PRSS2, RTN4RL1, SEMA3E, SIAT8D, SLC5A9, SLC40A1, SOX17, SPOCK3, TMOD1, TRPA1, TTN, AW166727, A1821586, BF941609, A1916532, BC034407, N63706 or AW772192, or any combination thereof. In some embodiments, the absence of cellular constituents, such as foregut markers Pdx1 and Albumin, can be used to reveal directed hindgut formation. In some embodiments, one or more (e.g. at least 1, 3) intestinal transcription factors CDX2, KLF5 or SOX9 can be used to represent intestinal development. In some embodiments, one or more of GATA4 or GATA6 protein expression can be used to represent intestinal development.

In some embodiments, morphological changes can be used to represent the progress of directed differentiation. In some embodiments, gut endoderm monolayer (e.g., mid-hindgut, hindgut, foregut, anterior foregut, or posterior foregut endoderm monolayer), or cells thereof, are subject to 3-dimensional culture conditions for maturation. In some embodiments, the gut endoderm monolayer matures to gastrointestinal organoids in a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or any number of days within a range defined by any two of the aforementioned number of days, for example, 1 to 40 days, 20 to 30 days, 30 to 40 days, or 1 to 20 days. In some embodiments, a highly convoluted epithelium surrounded by mesenchymal cells can be observed. In some embodiments, gastrointestinal organoids, epithelium, polarized columnar epithelium, mesenchyme, neuronal cells, or smooth muscle cells can be observed in a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, or any number of days within a range defined by any two of the aforementioned number of days, for example, 1 to 40 days, 20 to 30 days, 30 to 40 days, or 1 to 20 days.

In some embodiments, pluripotent stem cells are converted into gastrointestinal cell types via a “one step” process. For example, one or more molecules that can differentiate pluripotent stem cells into DE culture (e.g., Activin A) are combined with additional molecules that can promote directed differentiation of DE culture (e.g., CHIR99021 and FGF4) to directly treat pluripotent stem cells.

In some embodiments, pluripotent stem cells are prepared from somatic cells. In some embodiments, pluripotent stem cells are prepared from biological tissue obtained from a biopsy. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate. In some embodiments, PBMCs are grown on 0.1% gelatin.

In some embodiments, pluripotent stem cells are prepared from PBMCs by viral transduction. In some embodiments, PBMCs are transduced with Sendai virus, lentivirus, adenovirus, or adeno-associated virus, or any combination thereof. In some embodiments, PBMCs are transduced with Sendai virus comprising expression vectors for Oct3/4, Sox2, Klf4, or L-Myc, or any combination thereof. In some embodiments, PBMCs are transduced with one or more viruses at an MOI that is, is about, is at least, is at least about, is not more than, or is not more than about, 0, 0.1, 0.5, 1.0, 1.5, 2.0, 2.5, 3.0, 3.5, 4.0, 4.5, or 5.0 MOI, or any MOI within a range defined by any two of the aforementioned MOIs, for example, 0 to 5.0, 1.0 to 4.0, 2.0 to 3.0, 0 to 3.0, or 1.0 to 5.0. In some embodiments, after transduction, PBMCs express stem cell reprogramming factors. In some embodiments, after transduction, PBMCs are reprogrammed to iPSCs. In some embodiments, iPSCs are grown on a feeder cell substrate. In some embodiments, iPSCs are grown on a MEF feeder cell substrate. In some embodiments, iPSCs are grown on an irradiated MEF feeder cell substrate. In some embodiments, iPSCs are grown on 0.1% gelatin. In some embodiments, iPSCs are grown in RPMI 1640, DMEM, DMEM/F12, mTeSR 1, mTeSR Plus, DE Differentiation, Hindgut Endoderm Differentiation, Gut Base, or Complete Sato media.

In some embodiments, PSCs (e.g. ESCs or iPSCs) are cultured according to methods known in the art. In some embodiments, PSCs are expanded in an extracellular matrix, or mimetic or derivative thereof. In some embodiments, PSCs are expanded in Matrigel. In some embodiments, PSCs in culture are dissociated (e.g. using dispase) and plated onto Matrigel-coated plates for expansion. In some embodiments, PSCs are expanded in cell culture media comprising a ROCK inhibitor (e.g. Y-27632). In some embodiments, PSCs are expanded until at least 60%, 65%, 70%, 75%, 80%, 85%, 90%, or 95% confluence. In some embodiments, PSCs are differentiated into definitive endoderm cells. In some embodiments, PSCs are differentiated into definitive endoderm cells by contacting the PSCs with Activin A. In some embodiments, the PSCs are further contacted with one or more BMP signaling pathway activators, such as BMP4. In some embodiments, the PSCs are contacted with a concentration of each of the Activin A or the one or more BMP signaling pathway activators that is, is about, is at least, is at least about, is not more than, or is not more than about, 1, 2, 3, 4, 5, 6, 7, 8, 9, 10, 15, 20, 30, 40, 50, 60, 70, 80, 90, 100, 110, 120, 130, 140, 150, 160, 170, 180, 190, or 200 ng/mL, or any concentration within a range defined by any two of the aforementioned concentrations, for example, 1 to 200 ng/mL, 10 to 150 ng/mL, 1 to 100 ng/mL, or 100 to 200 ng/mL. In some embodiments, the iPSCs are differentiated into definitive endoderm in RPMI 1640, DMEM, DMEM/F12, mTeSR 1, mTeSR Plus, Day 1 DE Differentiation, Day 2 DE Differentiation, Day 3 DE Differentiation, Hindgut Endoderm Differentiation, Gut Base, or Complete Sato media. In some embodiments, DE differentiation media comprises one or more (e.g. at least 1, 2, 3, 4) of RPMI 1640, non-essential amino acids (NEAA), dialyzed fetal calf serum (dFCS), or Activin A, or any combination thereof. In some embodiments, Day 1 DE Differentiation media comprises 0% or about 0% dFCS, Day 2 DE Differentiation media comprises 0.2% or about 0.2% dFCS, and Day 3 DE Differentiation media comprises 2% or about 2% dFCS.

### Differentiation of Definitive Endoderm

Definitive endoderm represents the embryonic progenitor of many major organs, including the gastrointestinal tract (e.g. esophagus, lungs, thyroid, liver, pancreas, small intestine, large intestine). Methods of producing definitive endoderm cells from pluripotent stem cells (PSCs) include those conventionally known in the art. In some embodiments, the definitive endoderm is or has been differentiated from PSCs. In some embodiments, the definitive endoderm is or has been differentiated from embryonic stem cells (ESCs) or induced pluripotent stem cells (iPSCs). In some embodiments, the definitive endoderm or PSCs are derived from human. In some embodiments, the definitive endoderm is human definitive endoderm.

In some embodiments, the methods described herein for the production of one or more aggregated organoids comprise the step of differentiating definitive endoderm to a gut endoderm monolayer and gut spheroids. In some embodiments, the gut endoderm monolayer is adherent, for example, to the tissue culture plate or embodiments of the formation plate disclosed herein, and the gut spheroids are detached and suspended in a growth medium used to culture the definitive endoderm, gut endoderm monolayer, and gut spheroids. As used herein, gut endoderm refers to cells derived from the definitive endoderm which have undergone patterning to gastrointestinal lineages. In some embodiments, gut endoderm may comprise foregut endoderm, midgut endoderm, hindgut endoderm, or any combination thereof. In some embodiments, hindgut endoderm as used herein encompasses both midgut and hindgut endoderm and signifies small intestine and large intestine organ lineages. During differentiation of definitive endoderm to gut endoderm, gut spheroids spontaneously form and detach from the gut endoderm monolayer as suspended cell masses. These gut spheroids exhibit early characteristic of organoids, particularly heterogenicity of the constituent cell population comprising both epithelial and mesenchymal cell lineages.

Disclosed herein are methods of differentiating definitive endoderm to the gut endoderm monolayer and gut spheroids. However, previously known methods may also be employed to produce the gut endoderm monolayer. Methods may be found, for example, in U.S. Pat. Nos. 9,719,068 and 10,174,289, and PCT Publications WO 2016/061464, WO 2017/192997, WO 2018/106628, WO 2018/200481, WO 2018/085615, WO 2018/085622, WO 2018/085623, WO 2018/226267, WO 2020/023245, each of which is hereby expressly incorporated by reference in its entirety. Methods previously described for differentiating definitive endoderm to gut spheroids can be considered synonymous to differentiating definitive endoderm to both a gut endoderm monolayer and gut spheroids, as the production of gut spheroids typically results in the simultaneous production of gut endoderm monolayer.

In some embodiments, the definitive endoderm is differentiated to the gut endoderm monolayer and gut spheroids by contacting the definitive endoderm with one or more FGF signaling pathway activators, one or more Wnt signaling pathway activators, or one or more BMP signaling pathway inhibitors, or any combination thereof (e.g. at least 1, 2, or 3). In some embodiments, the one or more FGF signaling pathway activators comprise one or more FGF proteins disclosed herein or known in the art. In some embodiments, the one or more FGF signaling pathway activators comprise FGF4. In some embodiments, the one or more Wnt signaling pathway activators comprise one or more Wnt proteins disclosed herein or known in the art. In some embodiments, the one or more Wnt signaling pathway activators comprise one or more GSK3 inhibitors. In some embodiments, the one or more Wnt signaling pathway activators comprise CHIR99021. In some embodiments, the one or more BMP signaling pathway inhibitors comprise any BMP signaling pathway inhibitors disclosed herein or known in the art. In some embodiments, the one or more BMP signaling pathway inhibitors comprise Noggin. In some embodiments, the definitive endoderm is further contacted with retinoic acid. In some embodiments, the definitive endoderm is further contacted with EGF. In some embodiments, each of the one or more FGF signaling pathway activators, one or more Wnt signaling pathway activators, one or more BMP signaling pathway inhibitors, retinoic acid, or EGF, if provided, is contacted at a concentration that is, is about, is at least, is at least about, is not more than, or is not more than about, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 10 to 1000 ng/mL, 50 to 500 ng/mL, 500 to 1000 ng/mL, or 10 to 200 ng/mL. In some embodiments, each of the one or more FGF signaling pathway activators, one or more Wnt signaling pathway activators, one or more BMP signaling pathway inhibitors, retinoic acid, or EGF, if provided, is contacted at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1 to 20 PM, 1 to 10 μM, 5 to 15 μM, or 10 to 20 μM. In some embodiments, the definitive endoderm is differentiated to the gut endoderm monolayer and gut spheroids by culturing the definitive endoderm for 1, 2, 3, 4, 5, 6, 7, 8, 9, 10, 11, 12, 13, 14, or 15 days.

In some embodiments, the definitive endoderm is differentiated to a foregut endoderm monolayer and foregut spheroids. In some embodiments, the gut endoderm monolayer is a foregut endoderm monolayer and the gut spheroids are foregut spheroids. In some embodiments, differentiating the definitive endoderm to the foregut endoderm monolayer and the foregut spheroids comprises contacting the definitive endoderm with one or more FGF signaling pathway activators, one or more Wnt signaling pathway activators, or one or more BMP signaling pathway inhibitors, or any combination thereof (e.g. at least 1, 2, or 3). In some embodiments, the one or more FGF signaling pathway activators comprise FGF4, the one or more Wnt signaling pathway activators comprise CHIR99021, or the one or more BMP signaling pathway inhibitors comprise Noggin, or any combination thereof (e.g. at least 1, 2, or 3).

In some embodiments, the definitive endoderm is differentiated to a hindgut endoderm monolayer and hindgut spheroids. In some embodiments, the gut endoderm monolayer is a hindgut endoderm monolayer and the gut spheroids are hindgut spheroids. In some embodiments, differentiating the definitive endoderm to the hindgut endoderm monolayer and the hindgut spheroids comprises contacting the definitive endoderm with one or more FGF signaling pathway activators, or one or more Wnt signaling pathway activators, or both. In some embodiments, the one or more FGF signaling pathway activators comprise FGF4, or the one or more Wnt signaling pathway activators comprise CHIR99021, or both. In some embodiments, differentiating the definitive endoderm further comprises contacting the definitive endoderm with one or more BMP signaling pathway activators, for example, one or more BMP proteins described herein or known in the art. In some embodiments, the definitive endoderm is differentiated to the gut endoderm monolayer and gut spheroids in hindgut endoderm differentiation medium. In some embodiments, hindgut endoderm differentiation medium comprises one or more (e.g. at least 1, 2, 3, 4, 5) of RPMI 1640, NEAA, dFCS, FGF4, or CHIR99021, or any combination thereof. In some embodiments, hindgut endoderm differentiation medium comprises 2% or about 2% dFCS, 500 ng/mL or about 500 ng/mL FGF4, or 3 μM or about 3 μM CHIR99021, or any combination thereof.

In some embodiments, the gut endoderm monolayer produced by any of the methods disclosed herein are distinguished from gut spheroids produced according to previous methods by the relative abundance of mesoderm and/or mesenchyme lineages. In some embodiments, culturing the gut endoderm monolayer results in an increase in mesoderm and/or mesenchyme. In some embodiments, the gut endoderm monolayer comprises a percentage of mesoderm and/or mesenchyme relative to the total number of cells that is, is about, is at least, is at least about, is not more than, or is not more than about, 1%, 2%, 3%, 4%, 5%, 6%, 7%, 8%, 9%, 10%, 11%, 12%, 13%, 14%, 15%, 16%, 17%, 18%, 19%, or 20% of the total number of cells, or any percentage within a range defined by any two of the aforementioned percentages, for example 1% to 20%, 1% to 10%, 10% to 20%, or 5% to 15%. In some embodiments, the gut endoderm monolayer comprises more mesoderm and/or mesenchyme relative to gut spheroids at the same stage of culture. In some embodiments, the gut endoderm monolayer comprises a number of mesoderm and/or mesenchyme that is, is about, is at least, is at least about, is not more than, or is not more than about, 1, 2, 3, 4, or 5 times the number of mesoderm and/or mesenchyme found in gut spheroids, or any number of times within a range defined by any two of the aforementioned number of times.

### Gut Endoderm Isolation and Dissociation

After differentiation of the definitive endoderm to the gut endoderm monolayer and gut spheroids, any one of the methods disclosed herein comprise separating the gut endoderm monolayer from the gut spheroids, which are both in a growth medium. Any method of separating adherent cells (e.g. the gut endoderm monolayer) and suspension cells (e.g. the gut spheroids) known in the art may be employed. For example, as a non-limiting embodiment, the growth medium and suspended gut spheroids are aspirated to leave the gut endoderm monolayer. In some embodiments, one or more (e.g. 1, 2, 3, 4, 5, 6, 7, 8, 9, or 10) wash steps may be performed to ensure that all or most of the gut spheroids have been removed. In some embodiments, the growth medium may be agitated gently to resuspend any settled gut spheroids. In another non-limiting embodiment, the gut endoderm monolayer and gut spheroids are subjected to a continuous flow condition (e.g. within a flow chamber or cell) of fresh growth medium or wash solution to continuously remove any detached and suspended gut spheroids while retaining the adherent gut endoderm monolayer.

Following separating the gut endoderm monolayer from the gut spheroids in any one of the methods disclosed herein, the methods further comprise dissociating the gut endoderm monolayer to a single cell suspension of gut endoderm cells. In some embodiments, the gut endoderm cells comprise foregut endoderm cells, or hindgut endoderm cells, or both. In some non-limiting embodiments, dissociating the gut endoderm monolayer comprises mechanically dissociating or enzymatically dissociating the gut endoderm monolayer, or both. In some embodiments, the gut endoderm monolayer is dissociated with a proteolytic and/or collagenolytic enzyme. In some embodiments, the gut endoderm monolayer is enzymatically dissociated with Accutase (StemCell Technologies), Accumax (StemCell Technologies), trypsin, trypsin/EDTA, collagenase, dispase, TrypLE Express (Thermo Fisher), TrypLE Select (Thermo Fisher), or any combination thereof. In some embodiments, the gut endoderm monolayer is mechanically dissociated by trituration, for example, with a pipette. In some embodiments, the single cell suspension of gut endoderm cells is filtered to remove any non-dissociated cell masses.

### Gut Endoderm Aggregation

After dissociation of the gut endoderm monolayer to the single cell suspension of gut endoderm cells in any one of the methods disclosed herein, the methods further comprise aggregating the single cell suspension of gut endoderm cells into one or more gut endoderm aggregates. In some embodiments, the one or more gut endoderm aggregates are, comprise, consist essentially of, or consist of one or more foregut endoderm aggregates. In some embodiments, the gut endoderm aggregates are, comprise, consist essentially of, or consist of one or more hindgut endoderm aggregates. In some non-limiting embodiments, aggregating the single cell suspension of gut endoderm cells into the one or more gut endoderm aggregates comprises one or more (e.g. at least 1, 2, or 3) of aggregating the single cell suspension in hanging drops, centrifuging the single cell suspension in a microwell culture plate, centrifuging the single cell suspension in a “v” or “u”-bottomed microwell culture plate, aggregating the single cell suspension using an orbital shaker, or centrifuging the single cell suspension in a formation plate, or any combination thereof. In some embodiments, centrifuging the single cell suspension may be substituted with allowing the single cell suspension to settle into aggregates by gravity. In some embodiments, the formation plate is one of the formation plates disclosed herein. In some embodiments, each of the one or more gut endoderm aggregates comprises a number of cells that is, is about, is at least, is at least about, is not more than, or is not more than about, 50, 100, 150, 200, 250, 300, 350, 400, 450, 500, 550, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 gut endoderm cells, or any number of gut endoderm cells within a range defined by any two of the aforementioned numbers, for example, 50 to 10000 cells, 50 to 4000 cells, 1000 to 10000 cells, or 1000 to 5000 gut endoderm cells. In some embodiments, after aggregation, the one or more gut endoderm aggregates are cultured in RPMI 1640, DMEM, DMEM/F12, mTeSR 1, mTeSR Plus, DE Differentiation, Hindgut Endoderm Differentiation, Gut Base, or Complete Sato media. In some embodiments, Gut Base media comprises one or more (e.g. at least 1, 2, 3, 4, 5, 6) of Advanced DMEM/F12, B27 supplement, insulin, N2 supplement, HEPES buffer, penicillin/streptomycin, or L-glutamine, or any combination thereof. In some embodiments, Complete Sato media comprise one or more (e.g. at least 1, 2, 3, 4) of Gut Base media, EGF, Noggin, or R-spondin, or any combination thereof. In some embodiments, Complete Sato media comprises 500 ng/mL or about 500 ng/mL recombinant human EGF, 100 ng/mL or about 100 ng/mL recombinant human Noggin, or 500 ng/mL or about 500 ng/mL recombinant human R-spondin, or any combination thereof. In some embodiments, any of the media disclosed herein (e.g. Complete Sato media) may be supplemented with a ROCK inhibitor. In some embodiments, the ROCK inhibitor is Y-27632. In some embodiments, the ROCK inhibitor is supplemented at 10 μM or about 10 μM.

In some embodiments of any of the methods disclosed herein, gut endoderm cells are aggregated using an orbital shaker. In some embodiments, a suspension of gut endoderm cells are placed on an orbital shaker in a 37° C. incubator. The motion imparted to the suspension by the shaker results in cells contacting each other and the formation of aggregates. In some embodiments, aggregates of the gut endoderm cells are formed within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours of shaking, or any time within a range defined by any two of the aforementioned times, for example, 1 to 24 hours, 24 to 48 hours, or 12 to 36 hours.

In some embodiments of any of the methods disclosed herein, gut endoderm cells are aggregated by allowing the cells to settle out of suspension by gravity.

In some embodiments of any of the methods disclosed herein, gut endoderm cells are aggregated by a hanging drop method. In some embodiments, this hanging drop method comprises spotting a drop of gut endoderm cells suspending in growth media upside down on a surface (e.g. of a cell culture plate) and allowing the cells to sink to the bottom of the drop to aggregate.

### Formation Plates

FIGS. 2A-C depict embodiments of a preparation of an exemplary gut endoderm monolayer for single cell dissociation and aggregation. In some embodiments, aggregation is performed in a formation plate, a microwell culture plate, a “v”-bottomed microwell culture plate, a “u”-bottomed microwell culture plate, or using an orbital shaker, or any combination thereof. In some embodiments, the formation plate is an Aggrewell plate (StemCell Technologies), or generally any other plate for aggregating cells in accordance with the methods described herein.

First, with respect to FIGS. 2A and 2B, in some embodiments, a plurality of induced pluripotent stem cells (14) is cultured within a biocompatible container (16) under conditions either described herein or known in the art to form a definitive endoderm (18). In some embodiments, the definitive endoderm (18) continues to be cultured under conditions either described herein or known in the art to differentiate into a gut endoderm monolayer and gut spheroids within the biocompatible container (16). In some embodiments, the gut endoderm monolayer is a foregut endoderm monolayer or hindgut endoderm monolayer, and the gut spheroids are foregut spheroids or hindgut spheroids, but any variation of gut endoderm monolayer and/or gut spheroids is contemplated. In some embodiments, gut endoderm monolayer is adherent to the biocompatible container (16), while the gut spheroids are detached and are in suspension in a growth medium contained within the biocompatible container (16). In some embodiments, the gut endoderm monolayer is separated from the gut spheroids by aspirating the growth medium and suspended gut spheroids from the biocompatible container (16). In some embodiments, the isolated gut endoderm monolayer is then dissociated into a single cell suspension of gut endoderm cells (10), as described herein and depicted in FIG. 2C. In some embodiments, the single cell suspension (10) is collected and subjected to aggregation according to any of the methods disclosed herein or known in the art to form one or more gut endoderm aggregates (20). In some embodiments, the one or more gut endoderm aggregates (20) are placed into the same or different biocompatible container (16) to culture the one or more gut endoderm aggregates into one or more aggregated organoids.

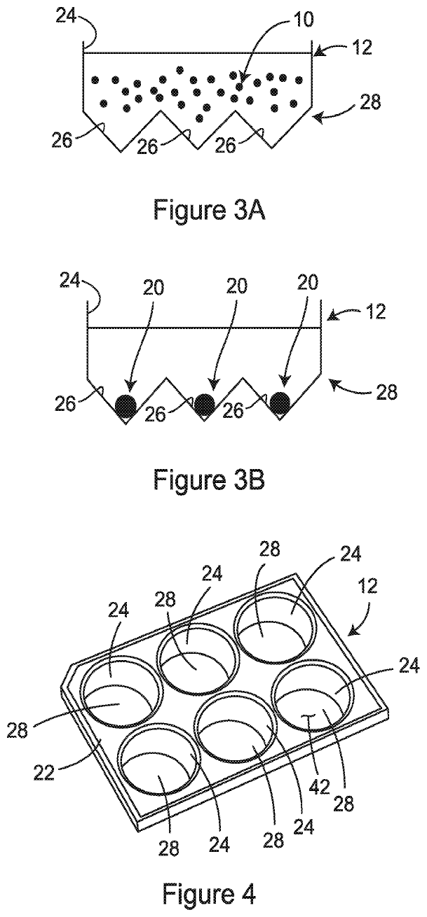

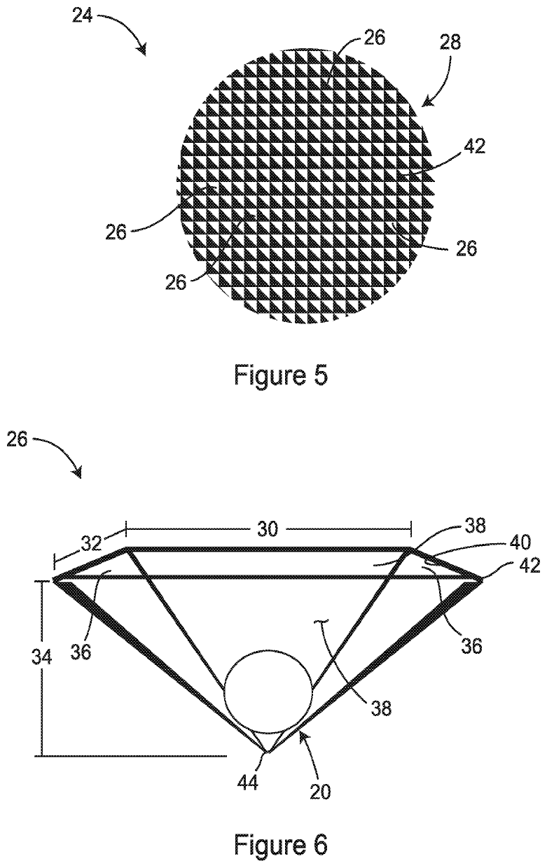

FIGS. 3A-6 depict embodiments of a formation plate (12). In some embodiments, the formation plate (12) has a base (22) and a plurality of wells (24). While the exemplary plate depicted in FIG. 4 has six wells, it will be appreciated that any number of wells (e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 100, 500, 1000, 2000, 5000 or more) may be similarly used. In some embodiments, each well (24) of the formation plate (12) comprises a plurality of microwells (26) along a bottom portion (28) thereof configured to receive and aggregate single cell suspension of gut endoderm cells (10) into a plurality of gut endoderm aggregates (20). With respect to FIG. 6, in some embodiments, each microwell (26) comprises a length (30), a width (32), and a depth (34). In some embodiments, the length (30) extends in a longitudinal direction that is, is about, is at least, is at least about, is not more than, or is not more than about, 100, 200, 300, 400, 500, 600, 700, 800, 900, or 1000 μm, or any length within a range defined by any two of the aforementioned lengths, for example, 100 to 1000 μm, 100 to 500 μm, 500 to 1000 μm, or 300 to 600 μm. In some embodiments, the length is defined between opposing longitudinal sidewalls (36) of the microwell (26). In some embodiments, the width (32) extends in a lateral direction that is, is about, is at least, is at least about, is not more than, or is not more than about, 100, 200, 300, 400, 500, 600, 700, 800, 900, or 1000 μm, or any width within a range defined by any two of the aforementioned widths, for example, 100 to 1000 μm, 100 to 500 μm, 500 to 1000 μm, or 300 to 600 μm. In some embodiments, the width is defined between opposing lateral sidewalls (38) of the microwell (26). In some embodiments, the depth (34) extends perpendicular to the longitudinal and lateral directions in a transverse direction that is, is about, is at least, is at least about, is not more than, or is not more than about, 50, 100, 150, 200, 250, 300, 350, 400, 450, or 500 μm, or any width within a range defined by any two of the aforementioned widths, for example, 50 to 500 μm, 50 to 300 μm, 300 to 500 μm, or 100 to 400 μm. In some embodiments, the depth (34) is defined between an opening (40) in an upper surface (42) of bottom portion (28) and a floor surface (44) of bottom portion (28). In some embodiments, each microwell (26) is defined between respective longitudinal sidewalls (36), lateral sidewalls (38), the opening (40), and the floor surface (44). In some embodiments, a lid may be included with plate (12) and configured to cover wells (24) so as to be encapsulated rather than open. In some embodiments, the formation plate (12) is not intended to be unnecessarily limited to the particular number, arrangement, or size of wells (24) and microwells (26) shown and described in any of the examples provided herein. In some embodiments, the formation plate is an Aggrewell plate (StemCell Technologies). In some embodiments, the Aggrewell plate is an Aggrewell 400 or Aggrewell 800 plate.

In some embodiments, to aggregate the single cell suspension (10), the microwell (26) tapers together from a relatively wider opening (40) toward a relatively narrower floor surface (44). In some embodiments, as depicted in FIG. 6, the opposing longitudinal sidewalls (36) taper toward each other from the opening (40) to the floor surface (44), while the opposing lateral sidewalls (38) similarly taper toward each other from the opening (40) to the floor surface (44). In some embodiments, gravity forces the single cells in the suspension downward in the transverse direction while the reactionary forces applied to the cells by the longitudinal and lateral sidewalls (36, 38) direct the cells inward toward each other to effectively gather and aggregate the single cells together. In some embodiments, such tapering allows aggregation of the cells as a 3-dimensional aggregate, which may be further encouraged with a centrifuge. In some embodiments, each microwell (26) receives a number of single cells that is, is about, is at least, is at least about, is not more than, or is not more than about, 50, 100, 200, 400, 600, 800, 1000, 1500, 2000, 2500, 3000, 3500, 4000, 4500, 5000, 6000, 7000, 8000, 9000, or 10000 single cells, or any number of cells within a range defined by any two of the aforementioned numbers, for example, 50 to 10000 cells, 50 to 4000 cells, 1000 to 10000 cells, or 1000 to 5000 cells.

In some embodiments, the longitudinal and lateral side walls (36, 38) have identical dimensions and define a void within the microwell (26) having a shape of an inverted pyramid. In some embodiments, the longitudinal sidewalls (36) and the lateral sidewalls (38) are planar and taper together toward the floor surface (44), which is essentially an inverted tip of the shape of a pyramid. In some embodiments, one or more of the longitudinal sidewalls (36), the lateral sidewalls (38), and the floor surface (44) are a continuous surface rather than intersecting at various edges. In some embodiments, the various sidewalls (36, 38) and floor surface (44) of the microwell (26) are not intended to be unnecessarily limited to the non-continuous, intersecting surfaces shown in some of the examples herein. In some embodiments, the void within the microwell (26) is shaped in other geometries that permit the collection and/or coalescence of containing cells. It is understood that one skilled in the art will be able to determine acceptable shapes for the microwells (26), which, for example, may include but are not limited to conical, dome, concave, elliptic, parabolic, and/or hyperbolic shapes. In some embodiments, the shape and size of the microwell is varied so as to be particularly configured for more effective growth for a particular population of single cells, such as endoderm or precursor cells associated with other tissues. In some embodiments, the invention is therefore not intended to be unnecessarily limited to the particular shape and dimensions of the formation plate (12) and/or microwells (26) shown in the Figures or for use with the particular cells discussed herein.

In some embodiments, each microwell (26) of any one of the formation plates described herein receives a number of single cells that is, is about, is at least, is at least about, is not more than, or is not more than about, 50, 100, 200, 400, 600, 800, 1000, 1500, 2000, 2500, 3000, 3500, 4000, 4500, 5000, 6000, 7000, 8000, 9000, or 10000 single cells, or any number of cells within a range defined by any two of the aforementioned numbers, for example, 50 to 10000 cells, 50 to 4000 cells, 1000 to 10000 cells, or 1000 to 5000 cells.

In some embodiments, the formation plate (12) has a single, unitary structure manufactured from a biocompatible material that inhibits attachment of cells to the formation plate (12) within the microwells (26) while allowing for development of the single cells (10) into the aggregates (20) as shown in the non-limited examples of FIGS. 3A-6. In some embodiments, the formation plate (12) is formed from a plurality of components with at least the surfaces of the microwell (26) being manufactured from a biocompatible material. In some embodiments, the biocompatible material comprises, consists essentially of, or consists of stainless steel, titanium, a polymeric organosilicon compound, polydimethylsiloxane (PDMS), glass, plastic, PVC, PE, PP, PMMA, PS, PTFE, nylon, polyurethane, PET, PES, hyaluronans, chitosan, sugars, ceramics, alumina, zirconia, bioglass, hydroxyapatite, or any combination thereof, or any other biocompatible material known in the art. In some embodiments, the formation plate (12) is sterile, resistant to adherence by tissues and/or cells, comprises a hydrophobic surface, comprises a feature that improves formation of the disclosed tissues and subsequent removal and/or use, or any combination thereof. In some embodiments, the formation plate (12) comprises one or more (e.g. at least 1, 3, 5, 10) small molecule compounds, activators, inhibitors, growth factors, nucleic acids, DNA, RNA, peptides, polypeptides, or proteins, or any combination thereof, that promotes growth and/or differentiation.

### Aggregated Organoids

In some embodiments, the methods disclosed herein further comprise culturing the one or more gut endoderm aggregates to produce the one or more aggregated organoids. In some embodiments, the one or more aggregated organoids described herein are or comprise esophageal organoids, gastric organoids, fundic gastric organoids, antral gastric organoids, hepatic organoids, intestinal organoids, or colonic organoids, or any combination thereof. In some embodiments, the one or more aggregated organoids are or comprise human esophageal organoids (HEOs), human gastric organoids (HGOs), human fundic gastric organoids (HFGOs), human antral gastric organoids (HAGOs), human hepatic organoids (HHOs), human intestinal organoids (HIOs), or human colonic organoids (HCOs), or any combination thereof. In some embodiments, after aggregating the single cell suspension of gut endoderm cells to one or more gut endoderm aggregates, the one or more gut endoderm aggregates are cultured for a short period of time that is, is about, is at least, is at least about, is not more than, or is not more than about, for example, 1, 2, 4, 6, 8, 10, 12, 14, 16, 18, 20, 22, 24, 26, 28, 30, 32, 34, 36, 38, 40, 42, 44, 46, 48, or 50 hours, or any period of time within a range defined by any two of the aforementioned times, such as 1 to 50 hours, 10 to 40 hours, 20 to 30 hours, 1 to 30 hours, or 24 to 50 hours, to collect, recover, and/or coalesce. In some embodiments, the one or more gut endoderm aggregates are dislodged or resuspended from the aggregation medium. For example, in some embodiments, where the one or more gut endoderm aggregates are aggregated in a microwell culture plate, “v” or “u”-bottomed microwell culture plate, or a formation plate, the one or more gut endoderm aggregates are dislodged from the microwells of the culture plate or formation plate (e.g. using a pipette to gently flow growth medium over the one or more gut endoderm aggregates, and aspirating the aggregates into the pipette tip). In some embodiments, the aggregation medium is washed with fresh growth medium, for example, Complete Sato medium, or other biocompatible aqueous solution to ensure that all of the aggregates are collected. In some embodiments, the one or more gut endoderm aggregates are collected in a container (e.g. a sterile tube) and allowed to settle by gravity. In some embodiments, centrifugation should not be used to collect the one or more gut endoderm aggregates, as this may cause the aggregates to fuse together. In some embodiments, after settling, the one or more gut endoderm aggregates are cultured under conditions to differentiate the one or more gut endoderm aggregates to the one or more aggregated organoids. For example, in some embodiments, after settling, any remaining growth medium is removed. In some embodiments, the one or more gut endoderm aggregates are contacted with a basement membrane or extracellular matrix, or a mimetic or derivative thereof. In some embodiments, the basement membrane or extracellular matrix, or mimetic or derivative thereof, comprises Matrigel. In some embodiments, the remaining growth medium is removed to reduce efficacy of the polymerization of the basement membrane or extracellular matrix, or the mimetic or derivative thereof. In some embodiments, the one or more gut endoderm aggregates are contacted with one or more growth factors, nutrients, vitamins, sugars, proteins, small molecules, agonists, antagonists, cytokines, signaling pathway activators or signaling pathway inhibitors to induce growth and maturation of the one or more gut endoderm aggregates into the one or more aggregated organoids. While conditions to differentiate the one or more gut endoderm aggregates to various different aggregated organoids are provided herein, other methods previously known to differentiate gut spheroids (e.g. foregut spheroids and/or hindgut spheroids) to respective organoids may also be used to differentiate the one or more gut endoderm aggregates in the same or similar fashion. Methods for organoid differentiation may be found, for example, in U.S. Pat. Nos. 9,719,068 and 10,174,289, and PCT Publications WO 2016/061464, WO 2017/192997, WO 2018/106628, WO 2018/200481, WO 2018/085615, WO 2018/085622, WO 2018/085623, WO 2018/226267, WO 2020/023245, each of which is hereby expressly incorporated by reference in its entirety.

In some embodiments, where the gut endoderm cells are foregut endoderm cells, the one or more gut endoderm aggregates are foregut endoderm aggregates and the one or more gut endoderm aggregates differentiate to one or more aggregated organoids of foregut lineage.

In some embodiments, the one or more aggregated organoids are or comprise one or more aggregated liver organoids. In some embodiments, culturing the one or more gut endoderm aggregates to form the one or more aggregated liver organoids comprises contacting the one or more gut endoderm aggregates with one or more (e.g. at least 1, 2, 3, 4, 5, 6) of one or more FGF signaling pathway activators, one or more BMP signaling pathway activators, retinoic acid, hepatocyte growth factor, dexamethasone, or Oncostatin M, or any combination thereof. In some embodiments, the one or more FGF signaling pathway activators comprise FGF2. In some embodiments, the one or more BMP signaling pathway activators comprise BMP4. In some embodiments, the one or more aggregated liver organoids comprise liver epithelium and liver mesenchyme.

In any of the provided embodiments, each of the one or more FGF signaling pathway activators, one or more BMP signaling pathway activators, retinoic acid, hepatocyte growth factor, dexamethasone, or Oncostatin M, if provided, is contacted at a concentration that is, is about, is at least, is at least about, is not more than, or is not more than about, 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 1 to 1000 ng/mL, 50 to 500 ng/mL, 500 to 1000 ng/mL, or 1 to 200 ng/mL. In some embodiments, each of the one or more FGF signaling pathway activators, one or more BMP signaling pathway activators, retinoic acid, hepatocyte growth factor, dexamethasone, or Oncostatin M, if provided, is contacted at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM.

In some embodiments, the one or more aggregated organoids are or comprise one or more aggregated gastric organoids. In some embodiments, the one or more aggregated gastric organoids are or comprise one or more aggregated fundic gastric organoids or one or more aggregated antral gastric organoids, or both. In some embodiments, culturing the one or more gut endoderm aggregates to form the one or more aggregated antral gastric organoids comprises contacting the one or more gut endoderm aggregates with one or more (e.g. at least 1, 2, or 3) of EGF, retinoic acid, or one or more BMP signaling pathway inhibitors, or any combination thereof. In some embodiments, the one or more BMP signaling pathway inhibitors comprise Noggin. In some embodiments, the one or more aggregated gastric organoids comprise gastric epithelium and gastric mesenchyme. In some embodiments, the gastric epithelium of the one or more aggregated gastric organoids is CDH1+, CLDN18+, or MUC5AC+, or any combination thereof.

In any of the provided embodiments, each of the EGF, retinoic acid, or one or more BMP signaling pathway inhibitors, if provided, is contacted at a concentration that is, is about, is at least, is at least about, is not more than, or is not more than about, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 10 to 1000 ng/mL, 50 to 500 ng/mL, 500 to 1000 ng/mL, or 10 to 200 ng/mL. In some embodiments, each of the EGF, retinoic acid, or one or more BMP signaling pathway inhibitors, if provided, is contacted at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1 to 20 μM, 1 to 10 μM, 5 to 15 μM, or 10 to 20 μM.

In some embodiments, where the gut endoderm cells are hindgut endoderm cells, the one or more gut endoderm aggregates are or comprise one or more hindgut endoderm aggregates and the one or more gut endoderm aggregates differentiate to one or more aggregated organoids of hindgut lineage.

In some embodiments, the one or more aggregated organoids are or comprise one or more aggregated intestinal organoids. In some embodiments, culturing the one or more gut endoderm aggregates to form the one or more aggregated intestinal organoids comprises contacting the one or more gut endoderm aggregates with one or more (e.g. at least 1, 2, or 3) of EGF, one or more Wnt signaling pathway activators, or one or more BMP signaling pathway inhibitors, or any combination thereof. In some embodiments, the one or more Wnt signaling pathway activators comprise R-spondin, or the one or more BMP signaling pathway inhibitors comprise Noggin, or both. In some embodiments, the one or more aggregated intestinal organoids comprise intestinal epithelium and intestinal mesenchyme. In some embodiments, the intestinal epithelium of the one or more aggregated intestinal organoids is CDH1+, CDX2+, E-cad+, or any combination thereof. In some embodiments, the intestinal mesenchyme of the one or more aggregated intestinal organoids is FOXF1+, CDX2+, Emilin+, or any combination thereof. In some embodiments, the intestinal epithelium of the one or more aggregated intestinal organoids exhibits proximal intestinal markers. In some embodiments, the proximal intestinal markers comprise CDH17, or PDX1, or both. In some embodiments, the one or more aggregated intestinal organoids are transplanted into a recipient subject and undergoes maturation. In some embodiments, the matured one or more aggregated intestinal organoids comprise intestinal cell types. In some embodiments, the intestinal cell types comprise epithelial cells, goblet cells, enteroendocrine cells, or Paneth cells, or any combination thereof. In some embodiments, the epithelial cells are SI+, the goblet cells are Muc2+, the enteroendocrine cells are chromogranin A+, or the Paneth cells are lysozyme+, or any combination thereof.

In some embodiments, the one or more aggregated organoids are or comprise one or more aggregated colonic organoids. In some embodiments, culturing the one or more gut endoderm aggregates to form the one or more aggregated colonic organoids comprises contacting the one or more gut endoderm aggregates with one or more (e.g. at least 1, 2, 3) of EGF, one or more Wnt signaling pathway activators, or one or more BMP signaling pathway activators, or any combination thereof. In some embodiments, the one or more Wnt signaling pathway activators comprise R-spondin, or the one or more BMP signaling pathway activators comprise BMP2, or any combination thereof. In some embodiments, the one or more aggregated colonic organoids comprise colonic epithelium and colonic mesenchyme. In some embodiments, the colonic epithelium is CDH1+ or SATB2+, or both.

In any of the provided embodiments, each of the EGF, one or more Wnt signaling pathway activators, one or more BMP signaling pathway inhibitors, or one or more BMP signaling pathway activators, if provided, is contacted at a concentration that is, is about, is at least, is at least about, is not more than, or is not more than about, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 10 to 1000 ng/mL, 50 to 500 ng/mL, 500 to 1000 ng/mL, or 10 to 200 ng/mL. In some embodiments, each of the EGF, one or more Wnt signaling pathway activators, one or more BMP signaling pathway inhibitors, or one or more BMP signaling pathway activators, if provided, is contacted at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 PM, or any concentration within a range defined by any two of the aforementioned concentrations, for example, 1 to 20 μM, 1 to 10 μM, 5 to 15 μM, or 10 to 20 μM. In some embodiments, the gut endoderm aggregates are cultured in Complete Sato media. In some embodiments, the Complete Sato media is supplemented with a ROCK inhibitor. In some embodiments, the ROCK inhibitor is Y-27632. In some embodiments, the ROCK inhibitor is supplemented at 10 μM or about 10 μM.

In some embodiments, the one or more gut endoderm aggregates are cultured for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 days, or any number of days within a range defined by any two of the aforementioned number of days, for example, 1 to 50 days, 10 to 30 days, 20 to 40 days, 1 to 30 days, or 20 to 50 days, to form the one or more aggregated organoids.

In some embodiments, the resulting one or more aggregated organoids are used to study esophageal, gastric, hepatic, intestinal, or colonic function, including but not limited to drug screening, neurological function, microbiome interaction, or transplant, or any combination thereof. In some embodiments, the one or more aggregated organoids comprise a functional lumen. In some embodiments, the one or more aggregated organoids have the ability to further differentiate upon transplantation. In some embodiments, the one or more aggregated organoids grow to the fetal stage in vitro and, upon transplantation, further differentiate.

### Uniformity and Scalability of Gut Endoderm Aggregates and Aggregated Organoids

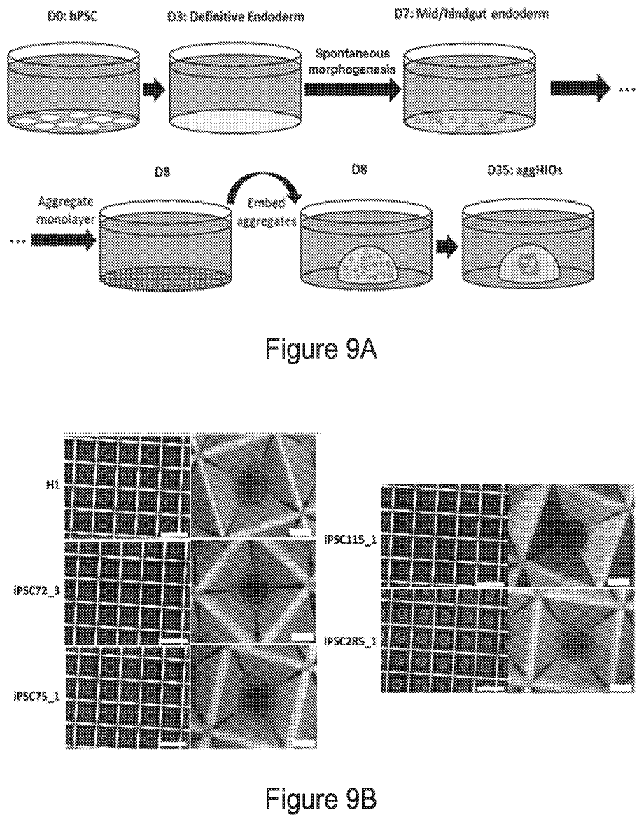

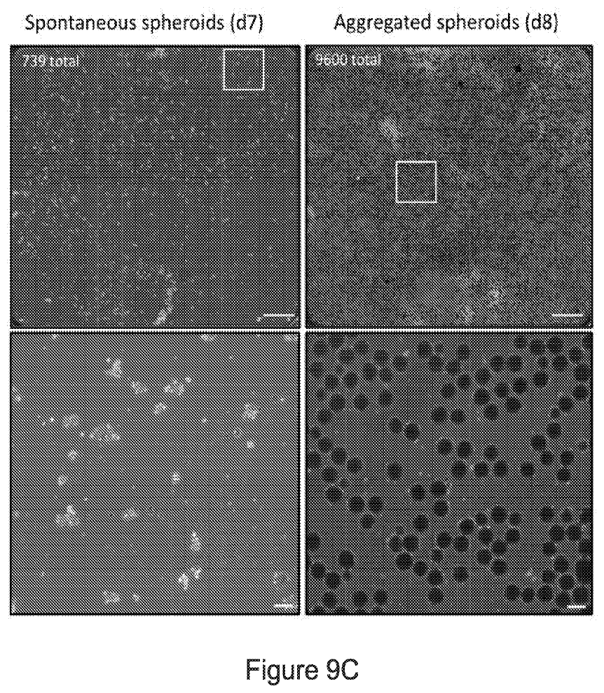

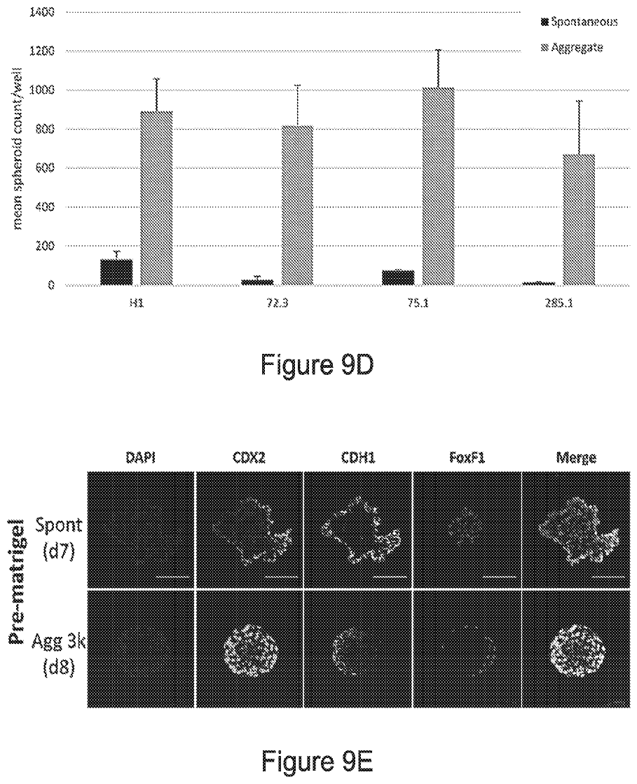

The methods disclosed herein in some embodiments permit the formation of many homogeneous or nearly homogeneous gut endoderm aggregates and/or resultant aggregated organoids. In some embodiments, the methods and the use of the aggregation mediums (e.g. any one of the formation plates disclosed herein) permit the formation of a plurality of gut endoderm aggregates. In some embodiments, the plurality of gut endoderm aggregates comprise a number of gut endoderm aggregates that is, is about, is at least, is at least about, is not more than, or is not more than about, 1000, 2000, 3000, 4000, 5000, 6000, 7000, 8000, 9000, 10000, 50000, 100000, 500000, or 1000000 gut endoderm aggregates, or any number of gut endoderm aggregates within a number defined by any two of the aforementioned number of gut endoderm aggregates, for example, 1000 to 1000000 gut endoderm aggregates, 5000 to 100000 gut endoderm aggregates, 1000 to 10000 gut endoderm aggregates, or 10000 to 1000000 gut endoderm aggregates. In some embodiments, the formation of homogeneous or nearly homogeneous gut endoderm aggregates and/or resultant aggregated organoids are defined by the plurality of gut endoderm aggregates and/or resultant aggregated organoids having reduced variance in at least one spatial dimension relative to gut endoderm spheroids and/or organoids produced from spheroids without aggregation. In some embodiments, the at least one spatial dimension comprises length, width, depth, volume, or surface area, or any combination thereof. In some embodiments, the gut endoderm aggregates and/or resultant aggregated organoids are spherical in geometry, and the at least one spatial dimension comprises radius, diameter, circumference, volume, or surface area, or any combination thereof. In some embodiments, the reduced variance in at least one spatial dimension comprises a diameter that is within ±10%, ±9%, ±8%, ±7%, ±6%, ±5%, ±4%, ±3%, ±2%, or ±1% from the average diameter of the plurality of gut endoderm aggregates and/or resultant aggregated organoids, or any diameter within a range defined by any two of the aforementioned diameters. In some embodiments, each of the plurality of gut endoderm aggregates and/or resultant aggregated organoids comprise a diameter that is within ±10%, ±90%, ±8%, ±7%, ±6%, ±5%, ±4%, ±3%, ±2%, or ±1% from the average diameter of the plurality of gut endoderm aggregates and/or resultant aggregated organoids, or any diameter within a range defined by any two of the aforementioned diameters. In some embodiments, the reduced variance in at least one spatial dimension comprises a volume that is within ±10%, ±9%, ±8%, ±7%, ±6%, ±5%, ±4%, 3%, 2%, or ±10% from the average volume of the plurality of gut endoderm aggregates and/or resultant aggregated organoids, or any volume within a range defined by any two of the aforementioned volumes. In some embodiments, each of the plurality of gut endoderm aggregates and/or resultant aggregated organoids comprise a volume that is within ±10%, 9%, ±8%, ±7%, ±6%, ±5%, ±4%, ±3%, ±2%, or ±1% from the average volume of the plurality of gut endoderm aggregates and/or resultant aggregated organoids, or any volume within a range defined by any two of the aforementioned volumes. In some embodiments, each of the plurality of gut endoderm aggregates and/or resultant aggregated organoids comprise both a diameter that is within ±10%, ±9%, ±8%, ±7%, ±6%, ±5%, ±4%, ±3%, ±2%, or ±1% from the average diameter of the plurality of gut endoderm aggregates and/or resultant aggregated organoids, or any diameter within a range defined by any two of the aforementioned diameters and a volume that is within ±10%, ±9%, ±8%, ±7%, ±6%, ±5%, ±4%, ±3%, ±2%, or ±1% from the average volume of the plurality of gut endoderm aggregates and/or resultant aggregated organoids, or any volume within a range defined by any two of the aforementioned volumes. For some embodiments, the reduced variance (i.e. uniformity) of the gut endoderm aggregates compared to spontaneously formed spheroids may be seen in FIGS. 9B, 9C, and 9E.

In any of the embodiments of the plurality of gut endoderm aggregates and/or resultant aggregated organoids, the plurality of gut endoderm aggregates and/or resultant aggregated organoids are derived from the same subject. In some embodiments, the subject is a mammal. In some embodiments, the subject is a human. In some embodiments, the subject has a disease, has had a disease previously, or is at risk of having a disease, or any combination thereof. In some embodiments, the disease is a gastrointestinal disease. In some embodiments, the plurality of gut endoderm aggregates and/or resultant aggregated organoids derived from the subject may be used for genetic testing or drug screening purposes. In some embodiments, the plurality of gut endoderm aggregates and/or resultant aggregated organoids derived from the subject may be used for large scale drug screening to identify effective therapeutics to reduce, ameliorate, or treat the disease of the subject. In some embodiments, the large-scale drug screening comprises testing multiple compounds each with a subpopulation of the plurality of gut endoderm aggregates and/or resultant aggregated organoids.

Also disclosed herein are embodiments of an aggregation medium comprising a plurality of microwells and plurality of gut endoderm aggregates. In some embodiments, the aggregation medium is any one of the microwell culture plates, “v” or “u”-bottomed microwell culture plates, or formation plate disclosed herein. In some embodiments, the plurality of gut endoderm aggregates is any one of the pluralities of gut endoderm aggregates disclosed herein, or the one or more gut endoderm aggregates disclosed herein. In some embodiments, the plurality of gut endoderm aggregates is any one of the pluralities of gut endoderm aggregates produced by any one of the methods disclosed herein, or the one or more gut endoderm aggregates produced by any one of the methods disclosed herein. In some embodiments, each of the plurality of microwells comprises 1, 2, 3, 4, 5, 6, 7, 8, 9, or 10 gut endoderm aggregates of the plurality of gut endoderm aggregates. In some embodiments, each of the plurality of microwells comprises a single gut endoderm aggregate of the plurality of gut endoderm aggregates.

### Transplantation and Methods of Treatment

In some embodiments, the methods disclosed herein comprise the additional step of transplanting any one or more of the aggregated organoids disclosed herein into a recipient subject. In some embodiments, the recipient subject is a mammal. In some embodiments, the recipient subject is a human. In some embodiments, the recipient subject is the subject from which the definitive endoderm, or precursor pluripotent stem cells, is derived. In some embodiments, the one or more aggregated organoids are derived from definitive endoderm or PSCs isolated from the recipient subject. In some embodiments, when transplanted into the recipient subject, the one or more aggregated organoids exhibit greater engraftment, maturation, growth, or any combination thereof compared to non-aggregated organoids known in the art.

In some embodiments, the one or more aggregated organoids as described herein are transplanted into a recipient subject, for example, as a treatment or an experimental model, as described herein. In some embodiments, the transplant is performed after culturing the organoid for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days of culture within a range defined by any two of the aforementioned days, for example, 1 to 50 days, 10 to 40 days, 20 to 30 days, 1 to 30 days, or 20 to 50 days. In some embodiments, the one or more aggregated organoids are mature enough for transplantation and/or study a number of days before organoids prepared by other methods known in the art are at the same or similar mature state, wherein the number of days is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 15 days, 1 to 15 days, or 10 to 20 days. In some embodiments, the recipient subject is a mammal. In some embodiments, the recipient subject is an immunodeficient mammal. In some embodiments, the recipient subject is an immunodeficient mouse. In some embodiments, the recipient subject is a monkey, dog, hamster, or rat. In some embodiments, the recipient subject is an immunocompromised monkey, dog, hamster, or rat. In some embodiments, the recipient subject is a human. In some embodiments, the recipient subject is an immunocompromised human. In some embodiments, the recipient subject is an immunocompetent human. In some embodiments, the recipient subject is an immunocompetent human treated with immunosuppressants. In some embodiments, the recipient subject is an immunocompetent human and the aggregated organoid is autologous to the host organism. In some embodiments, the recipient subject is an immunocompetent human and the aggregated organoid is allogeneic to the host organism. In some embodiments, the recipient subject is a mammal that is in need of an organ transplant. In some embodiments, the recipient subject is a human that is in need of an organ transplant.

In some embodiments, the one or more aggregated organoids are implanted to the appropriate region in the recipient subject. In some embodiments, the one or more aggregated organoids grow in the recipient subject for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the one or more aggregated organoids grow larger or matures faster than in vitro aggregated organoids prepared at the same time. In some embodiments, the one or more aggregated organoids exhibit integration with the recipient subject tissue. In some embodiments, the one or more aggregated organoids comprise gastrointestinal cell lineages. In some embodiments, the one or more aggregated organoids develop gastrointestinal cell lineages spontaneously.

Described herein are methods of treating a subject having compromised organ function, or ameliorating or inhibiting a detrimental organ disorder in a subject in need thereof. In some embodiments, the methods comprise transplanting or engrafting one or more aggregated organoids into the subject. In some embodiments, the one or more aggregated organoids are the one or more aggregated organoids of any one of the methods described herein. In some embodiments, the one or more aggregated organoids are or comprise one or more aggregated esophageal organoids, one or more aggregated gastric organoids, one or more aggregated fundic gastric organoids, one or more aggregated antral gastric organoids, one or more aggregated hepatic organoids, one or more aggregated small intestinal (intestinal) organoids, or one or more aggregated large intestinal (colonic) organoids, or any combination thereof. In some embodiments, the one or more aggregated organoids are autologous or allogeneic to the subject. In some embodiments, the one or more aggregated organoids are prepared from induced pluripotent cells obtained or derived from the subject. In some embodiments, the subject is in need of an organ transplant. In some embodiments, the one or more aggregated organoids are transplanted or engrafted as one or more whole aggregated organoids. In some embodiments, the transplant site is an organ tissue.

Also described herein is any one or more of the aggregated organoids produced by any one of the methods disclosed herein. Furthermore, in some embodiments are one or more aggregated organoids for use in restoring organoid function in a subject in need thereof. In some embodiments, the one or more aggregated organoids are the one or more aggregated organoids described herein. In some embodiments, the one or more aggregated organoids are the one or more aggregated organoids produced by any one of the methods described herein.

### Non-Limiting Methods of Producing Aggregated Organoids

Disclosed herein are methods of producing one or more aggregated organoids. In some embodiments, the methods comprise differentiating definitive endoderm to a gut endoderm monolayer and gut spheroids, separating the gut endoderm monolayer from the gut spheroids, dissociating the gut endoderm monolayer to a single cell suspension of gut endoderm cells, aggregating the single cell suspension of gut endoderm cells into one or more gut endoderm aggregates; and culturing the one or more gut endoderm aggregates to produce the one or more aggregated organoids. In some embodiments, the gut endoderm monolayer is adherent. In some embodiments, the separating step comprises aspirating the growth medium and suspended gut spheroids from the gut endoderm monolayer. In some embodiments, the dissociating step comprises enzymatically dissociating the gut endoderm monolayer. In some embodiments, the gut endoderm monolayer is enzymatically dissociated with Accutase, Accumax, trypsin, trypsin/EDTA, collagenase, dispase, TrypLE Express, or TrypLE Select, or any combination thereof. In some embodiments, the aggregating step comprises aggregating the single cell suspension in hanging drops, centrifuging the single cell suspension in a “v” or “u”-bottomed microwell culture plate, aggregating the single cell suspension using an orbital shaker, or centrifuging the single cell suspension in a formation plate, or any combination thereof. In some embodiments, centrifuging the single cell suspension may be substituted with allowing the single cell suspension to settle by gravity. In some embodiments, the formation plate is an Aggrewell plate. In some embodiments, each of the one or more gut endoderm aggregates comprise a number of cells that is, is about, is at least, is at least about, is not more than, or is not more than about, 50, 100, 150, 200, 250, 300, 350, 400, 450, 500, 550, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 gut endoderm cells, or any number of gut endoderm cells within a range defined by any two of the aforementioned numbers, for example, 50 to 10000 cells, 50 to 4000 cells, 1000 to 10000 cells, or 1000 to 5000 gut endoderm cells. In some embodiments, the culturing step comprises contacting the one or more gut endoderm aggregates with an extracellular matrix, or mimetic or derivative thereof. In some embodiments, the extracellular matrix, or mimetic or derivative thereof, comprises Matrigel. In some embodiments, the gut endoderm monolayer is a foregut endoderm monolayer and the gut spheroids are foregut spheroids. In some embodiments, the one or more aggregated organoids are or comprise one or more aggregated liver organoids. In some embodiments, the one or more aggregated organoids are or comprise one or more aggregated gastric organoids. In some embodiments, the one or more aggregated gastric organoids are or comprise one or more aggregated antral gastric organoids. In some embodiments, the gut endoderm monolayer is a hindgut endoderm monolayer and the gut spheroids are hindgut spheroids. In some embodiments, the one or more aggregated organoids are or comprise one or more aggregated intestinal organoids. In some embodiments, the one or more aggregated organoids are or comprise one or more aggregated colonic organoids.

Disclosed herein are methods of producing one or more aggregated organoids. In some embodiments, the methods comprise differentiating definitive endoderm to a gut endoderm monolayer and gut spheroids, separating the gut endoderm monolayer from the gut spheroids, dissociating the gut endoderm monolayer to a single cell suspension of gut endoderm cells, aggregating the single cell suspension of gut endoderm cells into one or more gut endoderm aggregates; and culturing the one or more gut endoderm aggregates to produce the one or more aggregated organoids. In some embodiments, the gut endoderm monolayer is adherent. In some embodiments, the separating step comprises aspirating the growth medium and suspended gut spheroids from the gut endoderm monolayer. In some embodiments, the dissociating step comprises enzymatically dissociating the gut endoderm monolayer. In some embodiments, the gut endoderm monolayer is enzymatically dissociated with Accutase. In some embodiments, the aggregating step comprises centrifuging the single cell suspension in a formation plate, or any combination thereof. In some embodiments, centrifuging the single cell suspension may be substituted with allowing the single cell suspension to settle by gravity. In some embodiments, the formation plate is an Aggrewell plate. In some embodiments, each of the one or more gut endoderm aggregates comprise a number of cells that is, is about, is at least, is at least about, is not more than, or is not more than about, 1000, 1500, 2000, 2500, 3000, 3500, 4000, 4500, or 5000 gut endoderm cells, or any number of gut endoderm cells within a range defined by any two of the aforementioned numbers, for example, 1000 to 5000 cells, 2000 to 4000 cells, 1000 to 3000 cells, or 3000 to 5000 gut endoderm cells. In some embodiments, the culturing step comprises contacting the one or more gut endoderm aggregates with Matrigel. In some embodiments, the gut endoderm monolayer is a foregut endoderm monolayer and the gut spheroids are foregut spheroids. In some embodiments, the one or more aggregated organoids are or comprise one or more aggregated liver organoids. In some embodiments, the one or more aggregated organoid are or comprise one or more aggregated gastric organoids. In some embodiments, the one or more gastric organoid are or comprise one or more aggregated antral gastric organoids. In some embodiments, the gut endoderm monolayer is a hindgut endoderm monolayer and the gut spheroids are hindgut spheroids. In some embodiments, the one or more aggregated organoids are or comprise one or more aggregated intestinal organoids. In some embodiments, the one or more aggregated organoid are or comprise one or more aggregated colonic organoids.

In some embodiments of any of the methods disclosed herein, the gut endoderm monolayer is a foregut endoderm monolayer and the gut spheroids are foregut spheroids. In some embodiments, differentiating the definitive endoderm to the foregut endoderm monolayer and the foregut spheroids comprises contacting the definitive endoderm with one or more FGF signaling pathway activators, one or more Wnt signaling pathway activators, or one or more BMP signaling pathway inhibitors, or any combination thereof. In some embodiments, each of the one or more FGF signaling pathway activators, one or more Wnt signaling pathway activators, or one or more BMP signaling pathway inhibitors, is contacted at a concentration that is, is about, is at least, is at least about, is not more than, or is not more than about, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 10 to 1000 ng/mL, 50 to 500 ng/mL, 500 to 1000 ng/mL, or 10 to 200 ng/mL. In some embodiments, each of the one or more FGF signaling pathway activators, one or more Wnt signaling pathway activators, or one or more BMP signaling pathway inhibitors, is contacted at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1 to 20 μM, 1 to 10 μM, 5 to 15 μM, or 10 to 20 μM. In some embodiments, the one or more FGF signaling pathway activators comprise FGF4. In some embodiments, the FGF4 is provided at a concentration of 500 ng/mL or about 500 ng/mL. In some embodiments, the one or more Wnt signaling pathway activators comprise CHIR99021. In some embodiments, the CHR99021 is provided at a concentration of 3 μM or about 3 μM. In some embodiments, the one or more BMP signaling pathway inhibitors comprise Noggin. In some embodiments, the Noggin is provided at a concentration of 200 ng/mL or about 200 ng/mL. In some embodiments, the foregut endoderm monolayer is dissociated to a single cell suspension of foregut endoderm cells. In some embodiments, the single cell suspension of foregut endoderm cells is aggregated to one or more foregut endoderm aggregates. In some embodiments, the one or more foregut endoderm aggregates are cultured to produce one or more aggregated liver organoids, or one or more aggregated gastric organoids, or both.

In some embodiments of any of the methods disclosed herein, the one or more aggregated organoids are or comprise one or more aggregated liver organoids. In some embodiments, the one or more foregut endoderm aggregates are cultured to produce one or more aggregated liver organoids. In some embodiments, culturing the one or more gut endoderm aggregates to form the one or more aggregated liver organoids comprises contacting the one or more gut endoderm aggregates with one or more FGF signaling pathway activators, one or more BMP signaling pathway activators, retinoic acid, hepatocyte growth factor, dexamethasone, or Oncostatin M, or any combination thereof. In some embodiments, each of the one or more FGF signaling pathway activators, one or more BMP signaling pathway activators, retinoic acid, hepatocyte growth factor, dexamethasone, or Oncostatin M, if provided, is contacted at a concentration that is, is about, is at least, is at least about, is not more than, or is not more than about, 1, 2, 3, 4, 5, 6, 7, 8, 9, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 1 to 1000 ng/mL, 50 to 500 ng/mL, 500 to 1000 ng/mL, or 1 to 200 ng/mL. In some embodiments, each of the one or more FGF signaling pathway activators, one or more BMP signaling pathway activators, retinoic acid, hepatocyte growth factor, dexamethasone, or Oncostatin M, if provided, is contacted at a concentration that is, is about, is at least, is at least about, is not more than, or is not more than about, 0.01, 0.1, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 0.01 to 20 μM, 0.01 to 10 μM, 1 to 15 μM, or 10 to 20 μM.

In some embodiments of any of the methods disclosed herein, the one or more aggregated organoids are or comprise one or more aggregated gastric organoids. In some embodiments, the one or more foregut endoderm aggregates are cultured to produce the one or more aggregated gastric organoids. In some embodiments, the one or more aggregated gastric organoids are or comprise one or more aggregated antral gastric organoids. In some embodiments, culturing the one or more gut endoderm aggregates to form the one or more aggregated antral gastric organoids comprises contacting the one or more gut endoderm aggregates with EGF, retinoic acid, or one or more BMP signaling pathway inhibitors, or any combination thereof. In some embodiments, each of the EGF, retinoic acid, or the one or more BMP signaling pathway inhibitors is contacted at a concentration that is, is about, is at least, is at least about, is not more than, or is not more than about, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 10 to 1000 ng/mL, 50 to 500 ng/mL, 500 to 1000 ng/mL, or 10 to 200 ng/mL. In some embodiments, each of the EGF, retinoic acid, or the one or more BMP signaling pathway inhibitors is contacted at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 PM, or any concentration within a range defined by any two of the aforementioned concentrations, for example, 1 to 20 μM, 1 to 10 μM, 5 to 15 μM, or 10 to 20 μM. In some embodiments, the EGF is provided at a concentration of 100 ng/mL or about 100 ng/mL. In some embodiments, the retinoic acid is provided at a concentration of 2 μM or about 2 μM. In some embodiments, the one or more BMP signaling pathway inhibitors comprise Noggin. In some embodiments, the Noggin is provided at a concentration of 200 ng/mL or about 200 ng/mL.

In some embodiments of any of the methods disclosed herein, the gut endoderm monolayer is a hindgut endoderm monolayer and the gut spheroids are hindgut spheroids. In some embodiments, differentiating the definitive endoderm to the hindgut endoderm monolayer and the hindgut spheroids comprises contacting the definitive endoderm with one or more FGF signaling pathway activators, or one or more Wnt signaling pathway activators, or both. In some embodiments, each of the one or more FGF signaling pathway activators, or one or more Wnt signaling pathway activators, or both, is contacted at a concentration that is, is about, is at least, is at least about, is not more than, or is not more than about, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 10 to 1000 ng/mL, 50 to 500 ng/mL, 500 to 1000 ng/mL, or 10 to 200 ng/mL. In some embodiments, each of the one or more FGF signaling pathway activators, or one or more Wnt signaling pathway activators, or both, is contacted at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1 to 20 μM, 1 to 10 μM, 5 to 15 μM, or 10 to 20 μM. In some embodiments, the one or more FGF signaling pathway activators comprise FGF4. In some embodiments, the FGF4 is provided at a concentration of 500 ng/mL or about 500 ng/mL. In some embodiments, the one or more Wnt signaling pathway activators comprise CHIR99021. In some embodiments, the hindgut endoderm monolayer is dissociated to a single cell suspension of hindgut endoderm cells. In some embodiments, the single cell suspension of hindgut endoderm cells is aggregated to one or more hindgut endoderm aggregates. In some embodiments, the one or more hindgut endoderm aggregates are cultured to produce one or more aggregated intestinal organoids, or one or more aggregated colonic organoids, or both.

In some embodiments of any of the methods disclosed herein, the one or more aggregated organoids are or comprise one or more aggregated intestinal organoids. In some embodiments, culturing the one or more gut endoderm aggregates to form the one or more aggregated intestinal organoids comprises contacting the one or more gut endoderm aggregates with EGF, one or more Wnt signaling pathway activators, or one or more BMP signaling pathway inhibitors, or any combination thereof. In some embodiments, each of the EGF, one or more Wnt signaling pathway activators, or the one or more BMP signaling pathway inhibitors is contacted at a concentration that is, is about, is at least, is at least about, is not more than, or is not more than about, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 10 to 1000 ng/mL, 50 to 500 ng/mL, 500 to 1000 ng/mL, or 10 to 200 ng/mL. In some embodiments, each of the EGF, one or more Wnt signaling pathway activators, or the one or more BMP signaling pathway inhibitors is contacted at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1 to 20 μM, 1 to 10 μM, 5 to 15 μM, or 10 to 20 μM. In some embodiments, the EGF is provided at a concentration of 500 ng/mL or about 500 ng/mL. In some embodiments, the one or more Wnt signaling pathway activators comprise R-spondin. In some embodiments, the R-spondin is provided at a concentration of 500 ng/mL or about 500 ng/mL. In some embodiments, the one or more BMP signaling pathway inhibitors comprise Noggin. In some embodiments, the Noggin is provided at a concentration of 100 ng/mL or about 100 ng/mL.

In some embodiments of any of the methods disclosed herein, the one or more aggregated organoids are or comprise one or more aggregated colonic organoids. In some embodiments, culturing the one or more gut endoderm aggregates to form the one or more aggregated colonic organoids comprises contacting the one or more gut endoderm aggregates with EGF, one or more Wnt signaling pathway activators, or one or more BMP signaling pathway activators, or any combination thereof.

In some embodiments, each of the EGF, one or more Wnt signaling pathway activators, or the one or more BMP signaling pathway activators is contacted at a concentration that is, is about, is at least, is at least about, is not more than, or is not more than about, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, or 1000 ng/mL, or any concentration within a range defined by any two of the aforementioned concentrations, for example, 10 to 1000 ng/mL, 50 to 500 ng/mL, 500 to 1000 ng/mL, or 10 to 200 ng/mL. In some embodiments, each of the EGF, one or more Wnt signaling pathway activators, or the one or more BMP signaling pathway activators is contacted at a concentration that is, is about, is at least, is at least about, is not more than, or is not more than about, 1, 2, 3, 4, 5, 6, 7, 8, 9, 10, 11, 12, 13, 14, 15, 16, 17, 18, 19, or 20 μM, or any concentration within a range defined by any two of the aforementioned concentrations, for example, 1 to 20 μM, 1 to 10 μM, 5 to 15 μM, or 10 to 20 μM. In some embodiments, the EGF is provided at a concentration of 500 ng/mL or about 500 ng/mL. In some embodiments, the one or more Wnt signaling pathway activators comprise R-spondin. In some embodiments, the R-spondin is provided at a concentration of 500 ng/mL or about 500 ng/mL. In some embodiments, the one or more BMP signaling pathway activators comprise BMP2. In some embodiments, the BMP2 is provided at a concentration of 100 ng/mL or about 100 ng/mL.

In some embodiments of any of the methods disclosed herein, the gut spheroids are detached and suspended in a growth medium. In some embodiments of any of the methods disclosed herein, the definitive endoderm has been differentiated from pluripotent stem cells. In some embodiments of any of the methods disclosed herein, the definitive endoderm has been differentiated from embryonic stem cells or induced pluripotent stem cells. In some embodiments of any of the methods disclosed herein, the definitive endoderm is human definitive endoderm.

In some embodiments of any of the methods disclosed herein, the methods further comprise transplanting the one or more aggregated organoids to a recipient subject. In some embodiments, the recipient subject is a mammal. In some embodiments, the recipient subject is a human.

Also described herein are the one or more aggregated organoids produced by any one of the methods disclosed herein.

### EXAMPLES

Some aspects of the embodiments discussed herein are disclosed in further detail in the following examples, which are not in any way intended to limit the scope of the present disclosure. Those in the art will appreciate that many other embodiments also fall within the scope of the disclosure, as it is described herein and in the claims.

### Example 1. Culture of Human Pluripotent Stem Cells (hPSCs)

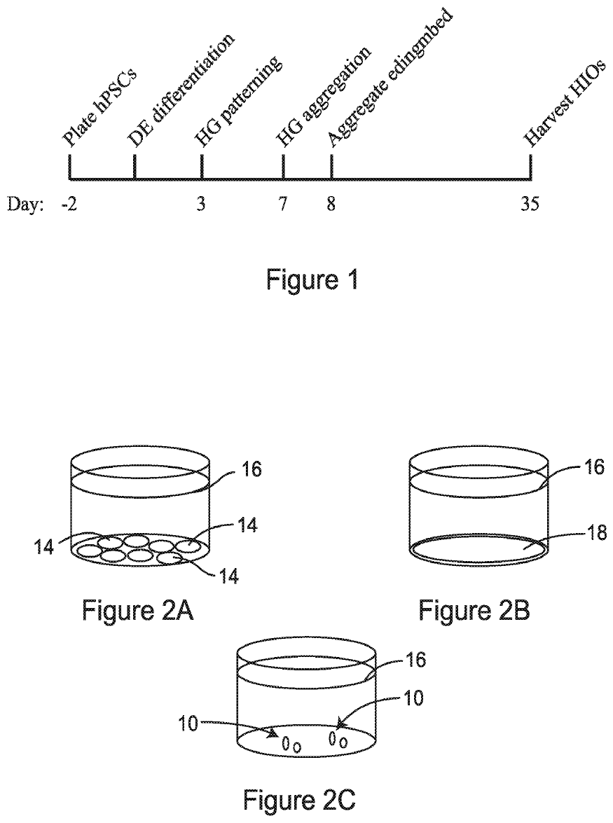

An exemplary schematic for the formation of aggregated organoids, such as aggregated human intestinal organoids (AggHIOs) is provided in FIG. 1.

Two days prior to differentiation of hPSCs to definitive endoderm (day −2), the hPSCs were cultured. A Matrigel-coated 24 well culture dish was prepared. 5 mL of Dispase solution (1 mg/mL; StemCell Technologies) was warmed to 37° C. If necessary, any areas of differentiation was removed from undifferentiated hPSCs. The media from each well containing hPSCs was carefully aspirated. 1 mL of pre-warmed Dispase solution was added to each well containing hPSCs and the cells were incubated at 37° C. until the colony edges appear slightly folded back. After about 4 minutes of Dispase incubation, it was confirmed that the edges of the hPSC colonies have started to lift away from the well. If colony edges have not lifted, the cells were incubated for an additional few minutes with regular checking until lifting was observed. During Dispase incubation, the Matrigel-coated dish was prepared by aspirating the Matrigel solution and adding 0.5 mL of fresh mTeSR1 medium (StemCell Technologies) to each well. The Matrigel-coated dish was not allowed to dry at any point. If necessary, the wells were aspirated and refilled with mTeSR1 one row at a time rather than all at once. The hPSC plate was removed from the incubator. The Dispase solution was gently aspirated and wells were washed at least three times with 2 mL of pre-warmed DMEM-F12 medium. It was important not to allow the plate to dry, and to avoid dislodging colonies while pipetting. Medium was gently dispensed onto the edge of each well. The DMEM-F12 wash was aspirated from each well, and 2 mL of warm mTeSR1 medium was added to each well. The colonies were carefully detached using a sterile disposable cell scraper. The mTeSR1 and cell aggregates were combined into a single well, taking care not to break up the cell aggregates excessively. Cells were triturated by carefully pipetting up and down once. If large aggregates are visible after a single trituration, the pipetting was repeated, checking aggregate size after each trituration. If necessary, this was done using a larger bore micropipette (e.g. p1000) to break up only the large aggregates. The hPSC aggregates were gently dispersed and 0.5 mL of the cell suspension was dispensed to each well of the Matrigel-coated 24 well plate. The newly plated cells were gently shaken back-and-forth and side-to-side to disperse the cells and then transferred to the incubator.

### Example 2. Definitive Endoderm (DE) Differentiation

On day 0 of culture, it was confirmed that the plated hPSCs (e.g. as described in Example 1) were evenly distributed at about 60-70% confluence and that the cells displayed standard undifferentiated morphology before starting differentiation. The mTeSR1 medium in each well was aspirated, taking care not to allow the cells to dry. 0.5 mL of day 1 DE differentiation medium (Table 1) was added per well. The cells were incubated at 37° C. and 5% CO₂ for 24 hours.

On day 1 of culture, the day 1 DE differentiation medium was discarded, and 0.5 mL of day 2 DE differentiation medium (Table 1) was added per well. The cells were incubated at 37° C. and 5% CO₂ for 24 hours.

On day 2 of culture, the day 2 DE differentiation medium was discarded, and 0.5 mL of day 3 DE differentiation medium (Table 1) was added per well. The cells were incubated at 37° C. and 5% CO₂ for 24 hours.

The DE differentiation media recipes provided in Table 1 are for making 1 mL of medium. The volumes may be scaled up as necessary. Final Activin A concentration on each day is 100 ng/mL. These media can be prepared in advanced and stored at 4° C., but it is preferable to prepare the day of use.

TABLE 1
DE differentiation media

| Material | Day 1 | Day 2 | Day 3 | Supplier; Catalog # |
|---|---|---|---|---|
| RPMI 1640 | 988 uL | 987 uL | 969 uL | Life Technologies; 11875 |
| 100x NEAA | 10 uL | 10 uL | 10 uL | Life Technologies; 11140- |
| | | | | 050 |
| Dialyzed fetal | 0 | 2 uL | 20 uL | Hyclone; SH30070 |
| calf serum | | (0.2%) | (2%) | |
| (dFCS) | | | | |
| Activin-A | 1 uL | 1 uL | 1 uL | Cell Guidance Systems; |
| (100 ng/μL) | | | | GFH6 |

### Example 3. Hindgut Endoderm Patterning

On day 3 of culture, the quality of the differentiated definitive endoderm was assessed based on the cells being in a flat, homogenous monolayer, and/or expression of definitive endoderm markers (e.g. Sox17, FoxA2, and/or CXCR4). The DE differentiation media was aspirated from each well, taking care not to allow the cells to dry. 0.5 mL of hindgut endoderm differentiation medium (Table 2) was added per well. The cells were incubated at 37° C. and 5% CO₂ for 24 hours.

On day 4 of culture, the previous hindgut endoderm differentiation medium was discarded, and 0.5 mL of fresh hindgut endoderm differentiation medium was added per well. The cells were incubated at 37° C. and 5% CO₂ for 24 hours.

On day 5 of culture, the previous hindgut endoderm differentiation medium was discarded, and 0.5 mL of fresh hindgut endoderm differentiation medium was added per well. The cells were incubated at 37° C. and 5% CO₂ for 24 hours. At this stage, the beginning of morphogenesis can be observed. There may be some detached spheroids in the wells. Care was taken not to aspirate these spheroids by tilting the plate while aspirating and leaving a small volume of media containing the spheroids in the well.

On day 6 of culture, the previous hindgut endoderm differentiation medium was discarded, and 0.5 mL of fresh hindgut endoderm differentiation medium was added per well. The cells were incubated at 37° C. and 5% CO₂ for 24 hours. At this stage, clear morphogenesis was observed. There may be some detached spheroids in the wells. Care was taken not to aspirate these spheroids by tilting the plate while aspirating and leaving a small volume of media containing the spheroids in the well.

On day 7 of culture (4 days of exposure to CHIR99021/FGF4), there was extensive morphogenesis and many detached spheroids were visible. The cells were processed for spheroid analysis as described in Example 4 or aggregation as described in Example 5.

The hindgut endoderm differentiation medium recipe provided in Table 2 are for making 1 mL of medium. The volumes may be scaled up as necessary.

TABLE 2
Hindgut endoderm differentiation medium recipe

| Material | Volume | | Final conc. | | Supplier; Catalog # |
|---|---|---|---|---|---|
| RPMI 1640 | 964.7 | uL | — | | Life Technologies; 11875 |
| 100x NEAA | 10 | uL | 1x | | Life Technologies; 11140- |
| | | | | | 050 |
| dFCS | 20 | uL | 2% | | Hyclone; SH30070 |
| recombinant | 5 | uL | 500 | ng/mL | R&D Systems; 235-F4 |
| human FGF4 | | | | | |
| (rhFGF4; | | | | | |
| 100 ng/uL) | | | | | |
| Chiron99021 | 0.3 | uL | 3 | μM | Stemgent; 04-0004-02 |
| (CHIR99021) | | | | | |
| (10 mM) | | | | | |

### Example 4. Existing Spheroid Protocols Results in Variability

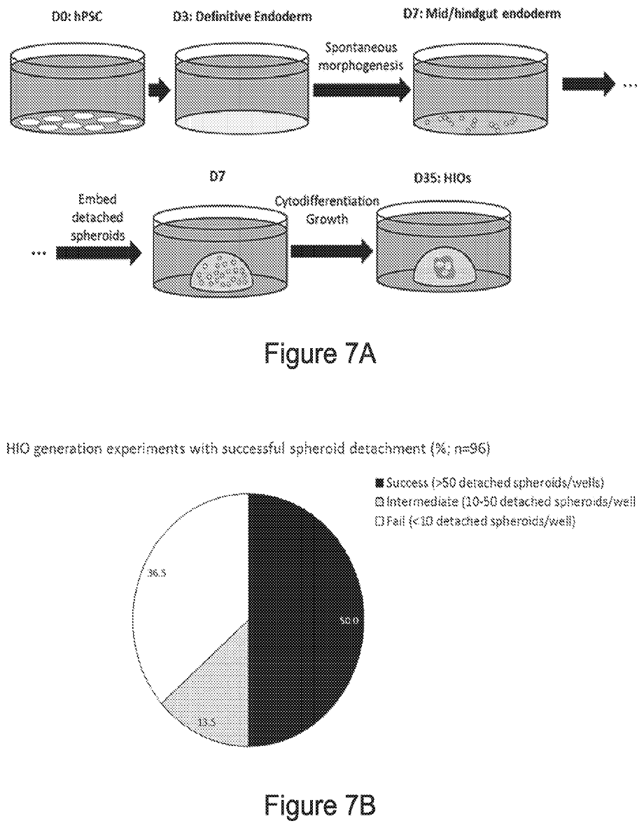

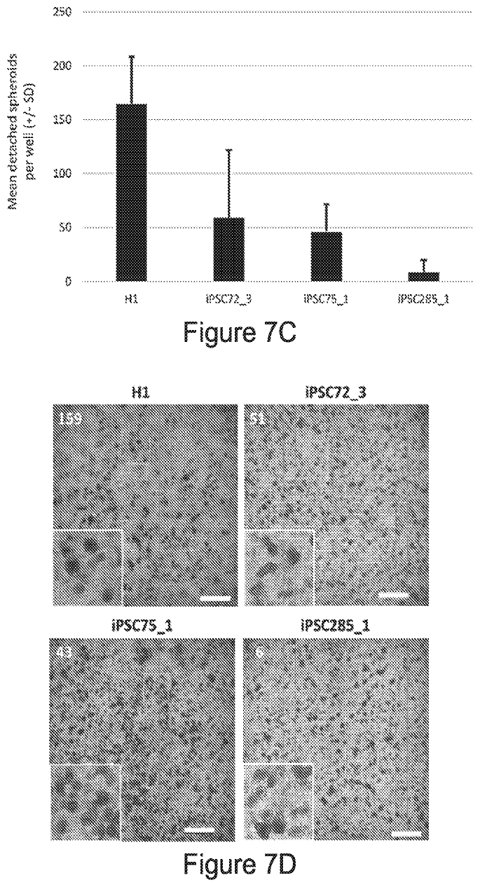

FIG. 7A describes a schematic for existing spheroid production directed to the formation of HIOs and is generally described in Examples 1-3. In summary, human pluripotent stem cells are first exposed to 100 ng/mL Activin A for 3 days to produce definitive endoderm (DE). DE is then exposed to a combination of 3 μM CHIR99021 and 500 ng/mL FGF4 for 4 days, during which patterning and morphogenesis to hindgut endoderm and spontaneous spheroid production occurs. At day 7, detached spheroids are collected from HGE monolayers, embedded in Matrigel, and cultured in 500 ng/mL EGF, 100 ng/mL Noggin, and 500 ng/mL R-spondin for 28 days. At day 35. HIOs are harvested and used for subsequent experimentation.

Spheroid production in multiple HIO generation experiments (n=96) using H1 human embryonic stem cells was assessed as Success (>50 detached spheroids/well); Intermediate (<50 detached spheroids/well); or Fail (no spheroid detachment). Scoring was performed by a single individual. As shown in FIG. 7B, there is significant variability in the formation of spheroids in separate replicates of the same protocol.

Spheroid production from H1 hESCs and 3 human iPSC lines (iPSC72_3, iPSC75_1, and iPSC285_1) was assessed using an existing protocol. Eight wells were plated per cell line and concurrently subjected to differentiation. At day 7, the number of detached spheroids per well was counted and an image of each well was captured. As shown in FIG. 7C, spheroid production varies among different PSC cell lines, having implications towards applications of personalized medicine. Exemplary images of spheroid production with the different PSC lines is shown in FIG. 7D. The number of detached spheroids in each well is indicated in the top-left corner of each condition. A line-to-line variability in detached spheroid number as well as robust morphogenesis but lack of spheroid detachment in some wells was observed.

### Example 5. Hindgut Endoderm Aggregation

Several approaches may be taken to aggregate hPSCs and cellular derivatives (e.g. foregut or hindgut endoderm). These include, but are not limited to, generation of hanging drops, centrifugation into 96-well or 384-well “v” or “u”-bottomed microwell culture plate, and aggregation of cells using an orbital shaker. The use of Aggrewells (StemCell Technologies) is described in this Example.

An Aggrewell 400 plate was prepared. Each well of the 24 well sized Aggrewell 400 plates can produce up to 1200 aggregates. The following is for a single well of an Aggrewell 400 plate. The amounts may be scaled up to prepare a sufficient number of wells/aggregates. 500 μL of Anti-Adherence Rising Solution (StemCell Technologies) was added to the well of the Aggrewell plate. The plate was centrifuged at 1300×g for 5 minutes in a swinging bucket rotor with a plate holder attachment. The plate was observed under a microscope to ensure that bubbles have been removed from the microwells. If bubbles remained, the centrifugation step was repeated. The Anti-Adherence Rinsing Solution was discarded. The well was rinsed with 2 mL of pre-warmed 37° C. Gut Base medium (Table 3). The Gut Base medium was discarded. 1 mL of pre-warmed 37° C. Complete Sato medium (Table 4) supplemented with 10 μM of Y-27632 was added to the well. The Aggrewell was kept in a 37° C. incubator until later use.

A single cell suspension of HGE cells was prepared. The media and any detached spheroids from the HGE endoderm tissue culture was discarded. 0.5 mL of pre-warmed 37° C. Accutase was added to each well, and the plate was incubated at 37° C. for about 5-10 minutes. The plate was monitored with a microscope to ensure that cells have been detached from the plate. If necessary, the cells may be incubated at 37° C. for an additional time period. Accutase may be substituted with other enzymatic dissociation reagents known in the art, such as trypsin, EDTA, TrypLE Express (Thermo Fisher), or TrypLE Select (Thermo Fisher), to prepare single cells. 0.5 mL of Complete Sato medium supplemented with 10 μM of Y-27632 was added to each well. Cells were gently dispersed with a pipette and transferred as single cells to a 15 mL centrifuge tube. The concentration of cells in the suspension was determined, and the number of cells to be used was calculated according to the following ratio: 1.2×10⁶ cells required per well of an Aggrewell 400 plate to form aggregates of 1,000 cells. This desired number of cells were transferred to a new centrifuge tube and centrifuged at 300×g for 5 minutes. The supernatant was discarded, and the cell pellet was resuspended in 1 mL of pre-warmed 37° C. Complete Sato medium supplemented with 10 μM Y-27632. The Aggrewell 400 plate previously prepared was removed from the incubator. Without removing the media already in the plate, the resuspended cells were transferred to the Aggrewell well. The cells were immediately mixed with a pipette to evenly distribute the cells throughout the well. The Aggrewell plate was centrifuged at 100×g for 3 minutes to capture the cells in the microwells. The plate is examined under a microscope to ensure that the cells are evenly distributed in the microwells. The Aggrewell plate was returned to the incubator overnight.

The Gut Base medium and Complete Sato medium recipes provided in Table 3 and 4 are for making 50 mL of medium. The volumes may be scaled up as necessary. The Gut Base medium may be stored at 4° C. for up to 2 weeks. For the Complete Sato medium, the EGF, Noggin, and R-spondin is added immediately before use.

TABLE 3
Gut Base Medium Recipe

| | | | Final | |
|---|---|---|---|---|
| Material | 50 mL | | conc | Supplier; Catalog # |
| Advanced DMEM/ | 46.75 | mL | — | Life Technologies; 12634028 |
| F12 | | | | |
| B27 + Insulin (50x) | 1 | mL | 1x | Life Technologies; 12587-10 |
| N2 Supplement | 500 | uL | 1x | Life Technologies; 17502-048 |
| (100x) | | | | |
| HEPES Buffer (1M) | 750 | uL | 15 mM | Life Technologies; 15630106 |
| Pen/Strep (100x) | 500 | uL | 1x | Life Technologies; 15140122 |
| L-Glutamine (100x) | 500 | uL | 1x | Life Technologies; 25030081 |

TABLE 4
Complete Sato Medium Recipe

| | | | Final | |
|---|---|---|---|---|
| Material | 50 ml | | conc | Supplier; Catalog # |
| Gut Base Media | 49.9 | mL | — | Life Technologies; |
| | | | | 12634028 |
| recombinant human | 50 | uL | 500 ng/mL | R&D Systems; 236-EG |
| EGF (rhEGF; 500 | | | | |
| ug/mL) | | | | |
| recombinant human | 50 | uL | 100 ng/mL | R&D Systems; 6057-NG |
| Noggin (rhNoggin; | | | | |
| 100 ug/mL) | | | | |
| recombinant human | 50 | uL | 500 ng/mL | R&D Systems; 4645-RS |
| R-Spondin1 | | | | |
| (rhRSpondin1; | | | | |
| 500 ug/mL) | | | | |

### Example 6. Harvesting and Embedding Aggregates

On day 8 of culture, for each well containing aggregates, the aggregates were gently dislodged from the microwells by pipetting and aspirated into the pipette tip. The collected aggregates were transferred to a sterile 15 mL centrifuge tube. To remove aggregates that remain in the well, 1 mL of pre-warmed 37° C. Complete Sato medium (without the ROCK inhibitor) was added and the collection process was repeated. This volume was combined with the previously collected aggregates. The collected aggregates (about 1200 per well of an Aggrewell 400) were allowed to settle to the bottom of the tube by gravity. After settling, as much supernatant as possible was removed from the aggregates. This removal step is important, as any liquid that remains will compromise the integrity of the Matrigel matrix upon resuspension. 200 μL of ice-cold Matrigel was added to the tube containing the aggregates. The solution was slowly pipetted up and down to evenly resuspend and mix the aggregates, while avoiding formation of air bubbles. 50 μL of the Matrigel/aggregate mixture was added to the center of the well of a 24 well tissue culture treated plate. Care was taken to keep the Matrigel in a single drop that does not touch the sides of the well, as if this happens, the Matrigel may flatten and the spheroids will likely adhere to the plastic. This plating process was repeated until all of the Matrigel/aggregate volume has been plated. The plate was quickly but carefully flipped upside down to prevent the spheroids from settling to the surface of the tissue culture plate. The plate was transferred to a 37° C. incubator for 20 minutes to facilitate Matrigel polymerization. Subsequently, 0.5 mL of pre-warmed 37° C. Complete Sato medium was added to each well. The medium was replaced every 3-4 days. The developing organoids should be passaged if the pH indicator changes rapidly or if the organoids appear very dense.

### Example 7. Aggregated Intestinal Organoids Resemble In Vivo Tissue with Greater Reproducibility

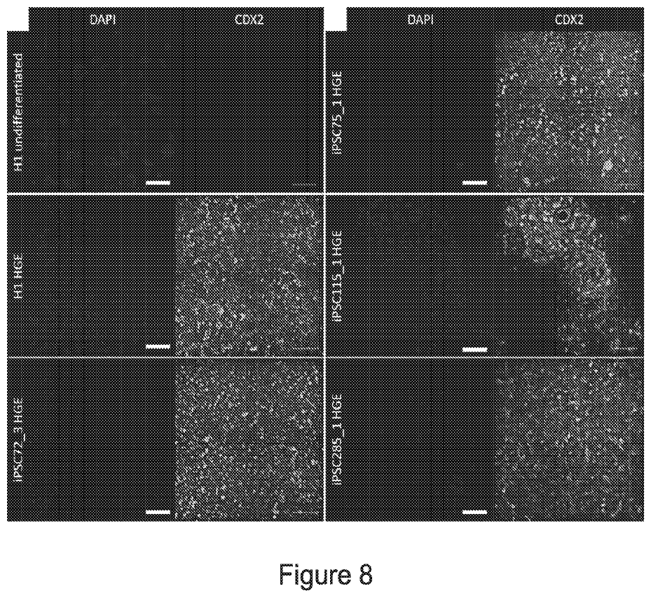

As a control, H1 hESCs and 4 human iPSC lines (iPSC72_3, iPSC75_1, iPSC115_1, and iPSC285_1) were subjected to differentiation. At day 7, formation of HGE was assessed by immunofluorescence for CDX2 (HGE marker) and DAPI (nuclei). Tile scans of 4 randomly selected areas of wells from each cell line are shown. As seen in FIG. 8, uniform expression of CDX2 was observed and demonstrates robust and efficient HGE production in all hPSC cell lines tested.

FIG. 9A describes a schematic for the production of gut endoderm aggregates and aggregated organoids, which is generally described in Examples 5-6. In summary, human pluripotent stem cells were first exposed to 100 ng/mL Activin A for 3 days to produce DE. DE is then exposed to a combination of 3 μM CHIR99021 and 500 ng/mL FGF4 for 4 days, during which patterning to hindgut endoderm and spontaneous spheroid production occurs. At day 7, regardless of the presence of detached spontaneous spheroids, a single cell suspension of HGE is prepared and subjected to aggregation using Aggrewell plates for 24 hours. Aggregates are then harvested from microwells, embedded in Matrigel and cultured in 500 ng/m L EGF, 100 ng/mL Noggin, and 500 ng/mL R-spondin for 28 days. At day 35, aggregated human intestinal organoids (aggHIOs) are harvested and used for subsequent experimentation.

H1 hESCs and 4 human iPSC lines (iPSC72_3, iPSC75_1, iPSC115_1, and iPSC285_1) were subjected to differentiation using an aggregation protocol. After 7 days of differentiation, HGE was dissociated into single cells using Accutase and aggregated for 24 hours in media containing 500 ng/mL EGF, 100 ng/mL Noggin, and 500 ng/mL R-spondin. After aggregation, images of cells in each Aggrewell were captured and are shown at 50× (left column; scale bar=500 μm) and 200× (right column; scale bar=100 μm). As seen in FIG. 9B, uniform aggregation of the gut endoderm cells is achievable with an Aggrewell formation plate.

H1 hESCs were subjected to differentiation using existing non-aggregation protocols. After 7 days of differentiation, spontaneously produced, detached spheroids were harvested, counted, and imaged using a Keyence BZ-X800 system. The remaining HGE (i.e. non-detached material) was then dissociated into single cells using Accutase and aggregated for 24 hours in media containing 500 ng/mL EGF, 100 ng/mL Noggin, and 500 ng/mL R-spondin. Aggregates were also then counted and imaged using the Keyence BZ-X800 system). Representative images are shown in FIG. 9C. Both an increased number of spheroids and more uniform size of spheroids produced from the non-detached monolayer using an aggregation method was observed.

The number of spheroids after culture of the gut endoderm aggregates in the formation plate was quantified and is shown in FIG. 9D. The number of detached spheroids produced per well using existing non-aggregation protocols was scored for H1 hESCs and 4 human iPSC lines (iPSC72_3, iPSC75_1, iPSC115_1, and iPSC285_1). In addition, the average number of aggregates formed per well from dissociated cells obtained from the non-detached HGE material in each experiment was determined. N=4 experiments.

Spontaneous detached (day 7) or aggregated (day 8) spheroids were fixed and subjected to immunostaining to identify intestinal epithelial cells (CDH1+/CDX2+) and intestinal mesenchymal cells (FoxF1/CDX2+). FIG. 9E shows representative images captured by confocal microscopy. Spheroids cultured from gut endoderm aggregates show robust patterning of intestinal epithelial and mesenchymal cells with a more uniform morphology.

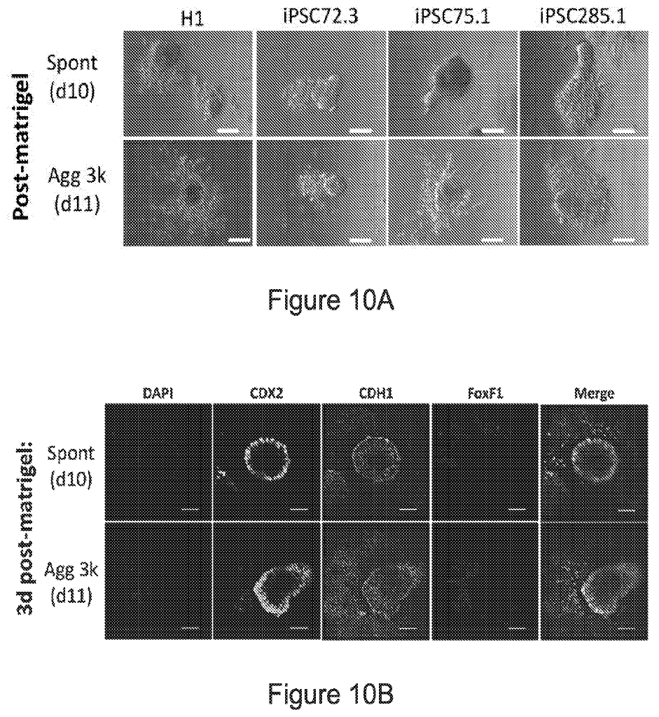

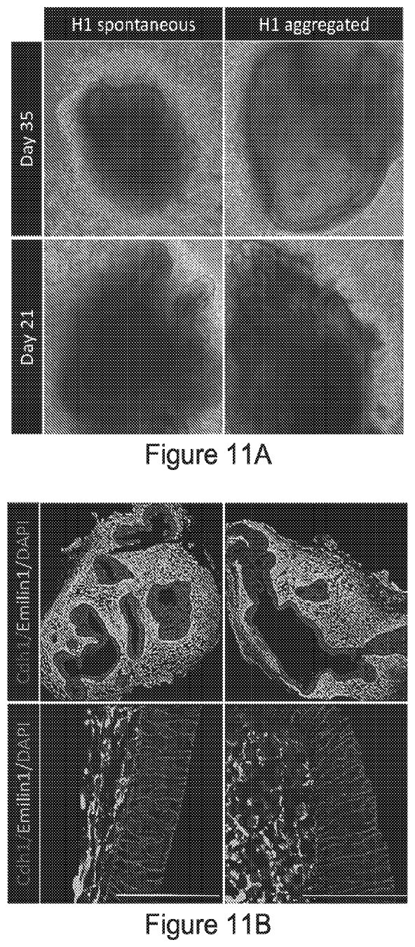

H1 hESCs and 4 human iPSC lines (iPSC72_3, iPSC75_1, iPSC115_1, and iPSC285_1) were subjected to differentiation to HGE. On day 7, detached spontaneous spheroids were directly embedded in Matrigel and non-detached cells were aggregated for 24 hours before embedding in Matrigel. After culture for 3 days in media containing 500 ng/mL EGF, 100 ng/mL Noggin and 500 ng/mL R-spondin, the gross morphology of embedded spheroids was assessed. As shown in FIG. 10A, both conditions were able to generate properly growing and maturing organoid precursors.

Spontaneous detached or aggregated spheroids were embedded in Matrigel. After 3 days of culture, spheroids were fixed and subjected to whole-mount immunostaining to identify intestinal epithelial cells (CDH1+/CDX2+) and intestinal mesenchymal cells (FoxF1/CDX2+). As shown in FIG. 10B, both conditions resulted in organoids comprising intact intestinal epithelium and mesenchyme.

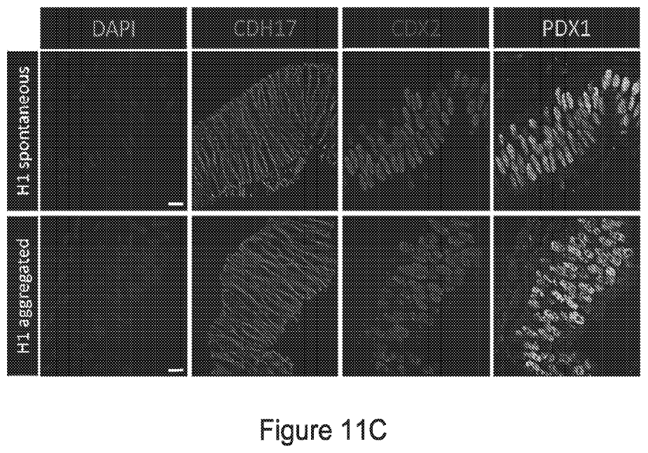

H1 hESCs were subjected to differentiation to HGE. At day 7, detached spontaneous spheroids were embedded in Matrigel and non-detached HGE was dissociated and aggregated for 24 hours before embedding in Matrigel. At days 18, 25, and 35, morphological analysis of organoids demonstrated that HIOs arising from both detached spheroids and aggregated HGE exhibited similar organoid growth and comprised discrete epithelial and mesenchymal layers (FIG. 11A).

At day 35, H1-derived HIOs and AggHIOs were harvested and subjected to co-immunofluorescence analysis for the presence of intestinal epithelia (CDX2+/E-cad+) and mesenchymal cells (Emilin1). As shown in FIG. 11B, both conditions resulted in well-formed CDX2+/E-cad+ epithelium and Emilin1+ mesenchyme.

At day 35, H1-derived HIOs and AggHIOs were harvested and subjected to immunofluorescence analysis for the presence of the proximal intestinal markers CDH17 and PDX1. All CDX2+ epithelial cells were positive for both CDH17 and PDX1 indicating proximal small intestinal patterning. As shown in FIG. 11C, both conditions resulted in patterning to CDH17+/PDX1+ proximal small intestine tissue.

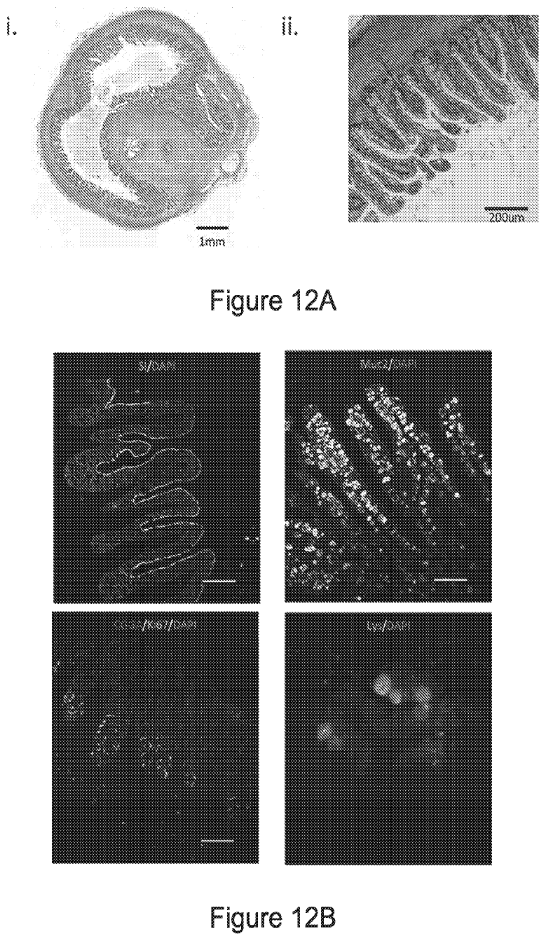

Day 35 AggHIOs were harvested and engrafted into the kidney capsule of immunodeficient mice. After 6 weeks, mice were euthanized, and engrafted AggHIOs were excised and subjected to histological analysis with hematoxylin/eosin (H&E) staining. As shown in FIG. 12A, the aggregated intestinal organoids are suitable for transplantation, and experience robust growth and maturation.

Transplanted AggHIOs were sectioned and subjected to immunofluorescence analysis for mature intestinal markers sucrase-isomaltase (SI; epithelia), Muc2 (goblet cells), chromogranin A (enteroendocrine cells), and lysozyme (Paneth cells). As shown in FIG. 12B, the transplanted aggregated intestinal organoids were positive for all tested intestinal cell markers.

### Example 8. Formation of Aggregated Gastric Organoids

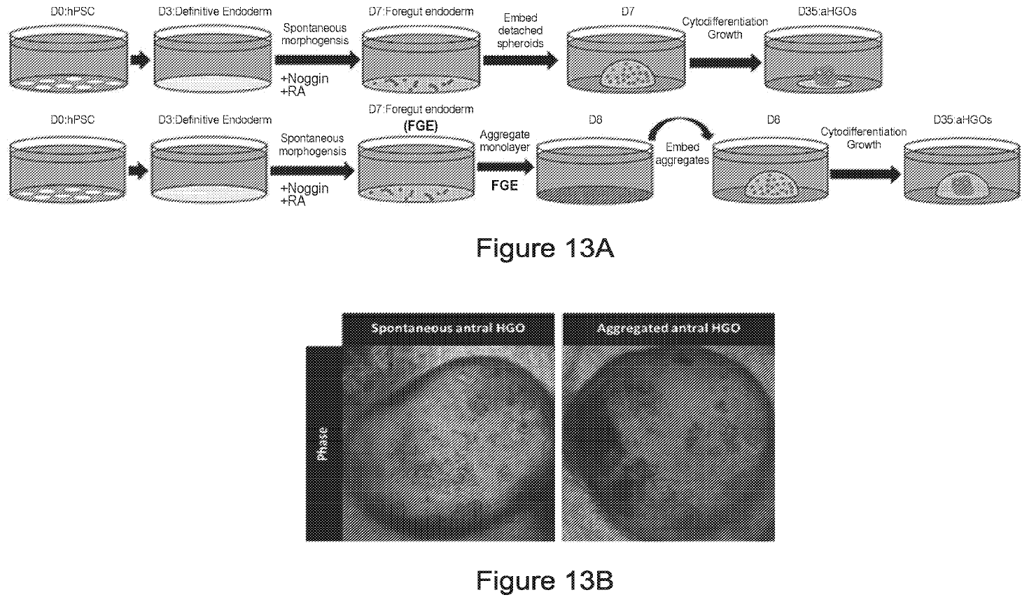

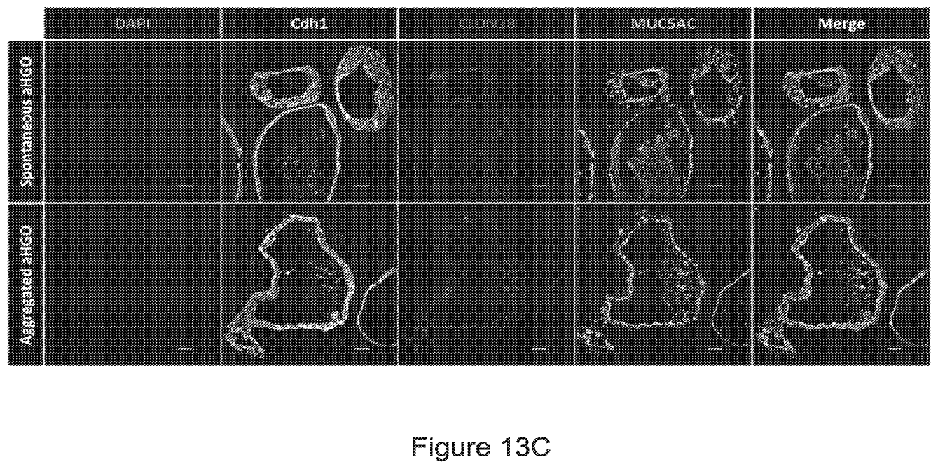

FIG. 13A depicts a schematic for the formation of aggregated antral gastric organoids. A comparison between existing methods and the aggregation method is provided. Top of FIG. 13A: method relying on spontaneous formation of antral organoids (e.g. as seen in McCracken et al. Nature. (2014) 516(7531):400-4)). Bottom of FIG. 13A: improved protocol incorporating a step for aggregation of foregut endoderm.

Antral organoids were generated from spontaneous detached spheroids and from spheroids generated from aggregated foregut endoderm. Representative images of aHGO morphology were taken at day 35 (FIG. 13B).

Day 35 aHGOs generated either from spontaneous spheroids or aggregated foregut endoderm were fixed, sectioned, and subjected to immunostaining for gastric epithelial markers CLDN18 and MUC5AC. Epithelial cells in aHGOs derived from both spontaneous and aggregated cells were uniformly Cdh1/CLDN18/MUC5AC positive (FIG. 13C).

### Example 9. Formation of Aggregated Colonic Organoids

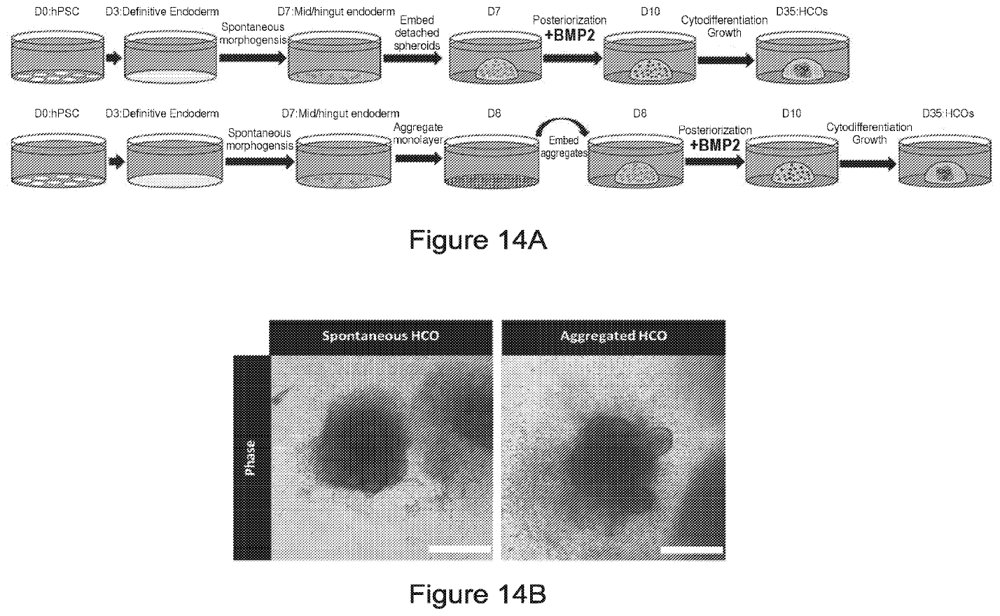

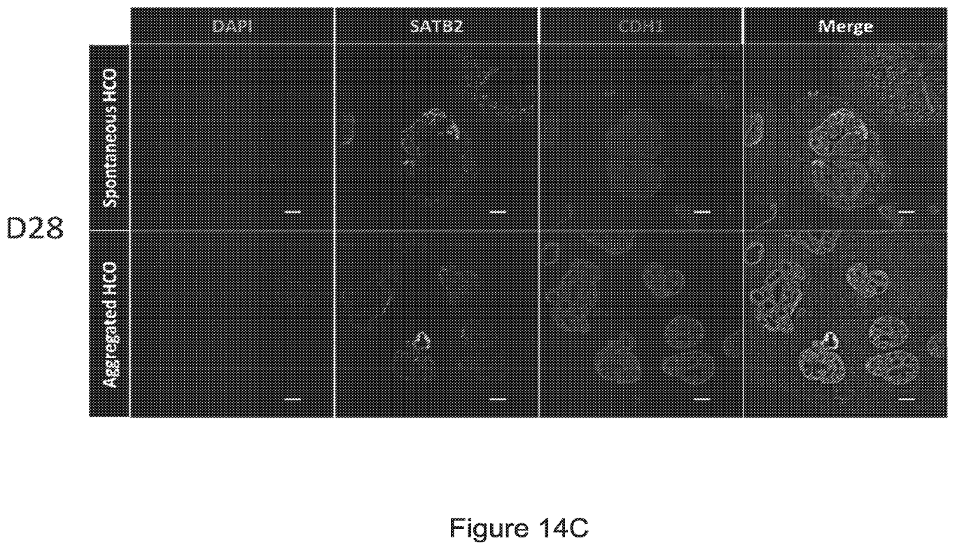

FIG. 14A depicts a schematic for the formation of aggregated colonic organoids. A comparison between existing methods and the aggregation method is provided. Top of FIG. 14A: method relying on spontaneous formation of colonic organoids. Bottom of FIG. 14A: improved protocol incorporating a step for aggregation of spheroids before posteriorization with BMP2.

HCOs were generated from spontaneous, detached spheroids and from spheroids generated from aggregated hindgut endoderm. Spheroids were then patterned to a posterior fate by exposure to BMP2 for 3 days. After 32 days, representative images of HCO morphology were captured (FIG. 14B).

Day 35 HCOs generated either from spontaneous spheroids or aggregated hindgut endoderm were fixed, sectioned and subjected to immunostaining for the colonic epithelial marker SATB2. CDH1+ epithelia in HCOs generated from spontaneous and aggregated hindgut endoderm exhibited similar numbers of SATB2 positive cells (FIG. 14C).

### Example 10. Formation of Aggregated Liver Organoids

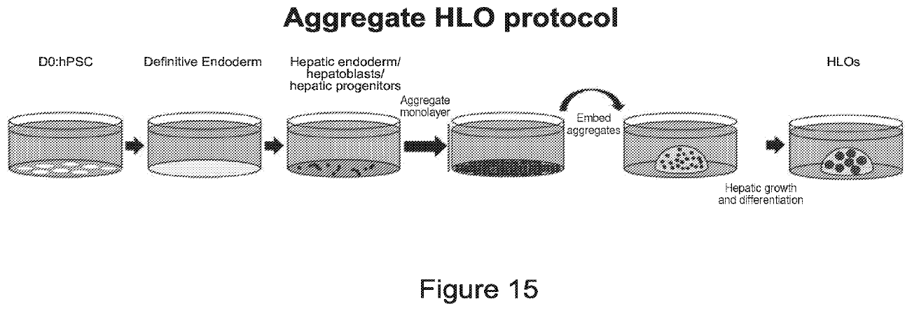

FIG. 15 depicts a schematic for the formation of aggregated liver organoids. HLOs are generated from spontaneous, detached spheroids and from spheroids generated from aggregated hindgut endoderm. Spheroids are then patterned to liver organoids by exposure to FGF2 (10-100 ng/mL), BMP4 (10-100 ng/mL), retinoic acid (2 μM), hepatocyte growth factor (10-20 ng/mL), dexamethasone (0.1 μM), and Oncostatin M (10-100 ng/mL).

### Example 11. Culturing of Gut Endoderm Monolayers Increase Mesoderm Populations

As conventional protocols for the differentiation of gut endoderm involve in vitro culturing of definitive endoderm with minimal growth factors, the resultant gut endoderm and downstream spheroids/organoids comprise ratios of other important cell types such as mesoderm and mesenchyme that is non-representative (i.e. less) than in vivo tissue. In normal development, the mesoderm and associated mesenchyme is important for proper cellular organization and tissue maturation. Accordingly, there is a need to increase mesoderm/mesenchymal populations in organoid compositions.

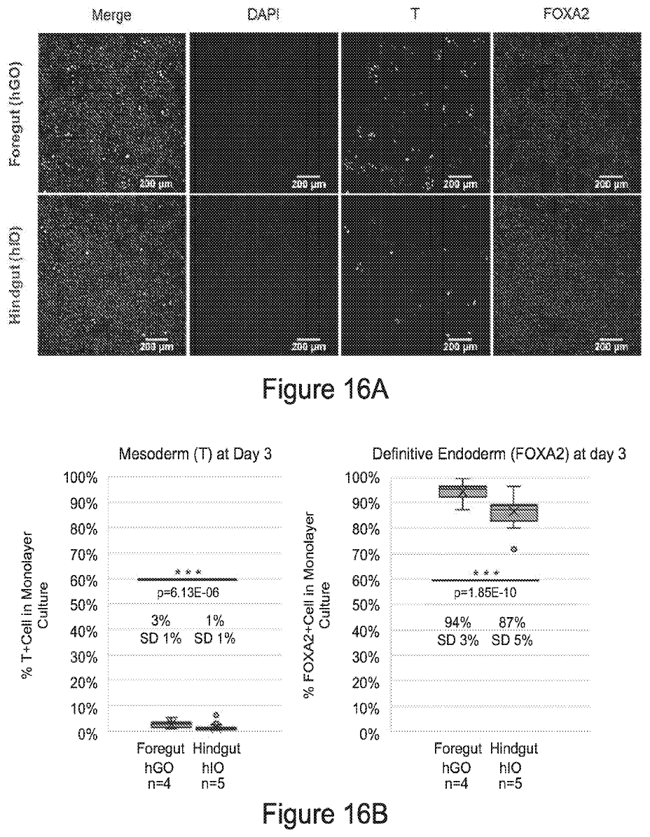

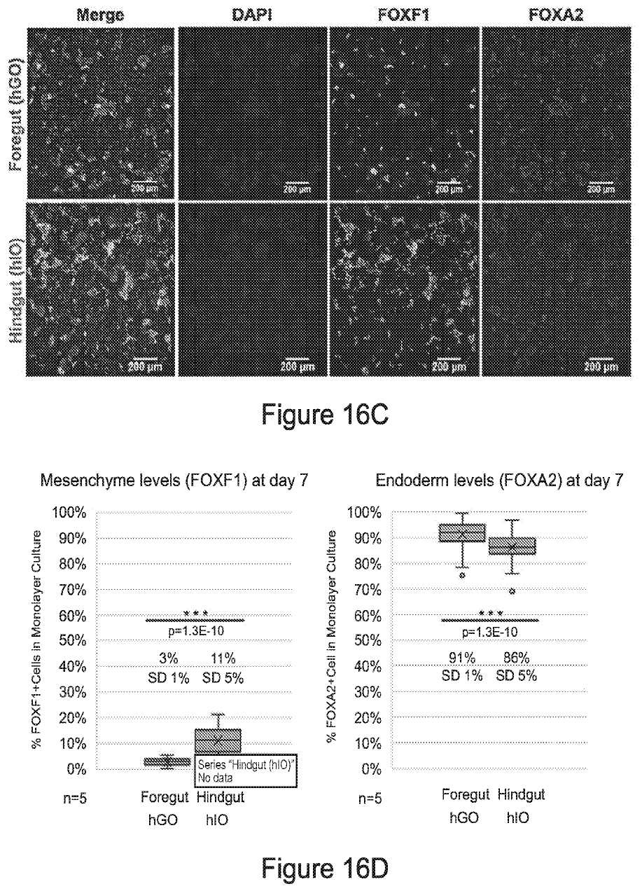

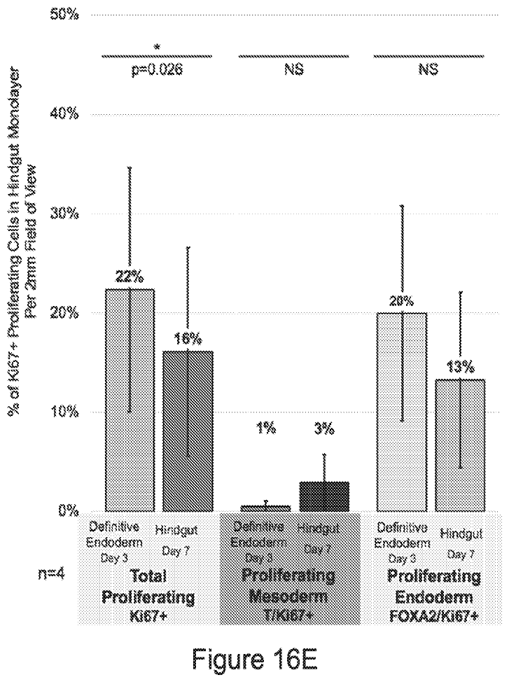

Day 3 foregut and hindgut endoderm monolayers were prepared according to the procedures explored in Examples 1-5 and 8. The monolayers were examined by immunofluorescence imaging staining for the mesoderm marker Brachyury (T) and the definitive endoderm marker FOXA2 (FIG. 16A). The images show that the monolayers are composed of mostly definitive endoderm with few mesoderm. The mesoderm and definitive endoderm populations of these gut endoderm monolayers were quantified (FIG. 16B). In both foregut and hindgut endoderm monolayers, there was only, on average, 3% and 1%, respectively, mesoderm cells, and 94% and 87%, respectively, definitive endoderm cells.

Like the Day 3 monolayer cultures, Day 7 foregut and hindgut endoderm monolayer cultures were prepared. As these cultures were further along in differentiation and gut patterning (including formation of spontaneous spheroids), mesenchyme cells, rather than mesoderm cells, were examined. The monolayers were stained with the mesenchyme marker FOXF1 and the endoderm marker FOXA2, and a general increase in the mesenchyme fraction was observed (FIG. 16C). The mesenchyme and endoderm populations were quantified (FIG. 16D). The foregut endoderm monolayer contained 3% mesenchyme and 91% endoderm, while the hindgut endoderm monolayer contained 11% mesenchyme and 86% endoderm. As observed, there is a significant increase in mesoderm/mesenchyme lineages after culturing of the hindgut endoderm monolayer.

Quantification of hindgut endoderm monolayers was repeated in an independent experiment. As seen in FIG. 16E, again, there is an increase in mesoderm/mesenchyme lineages in day 7 hindgut endoderm monolayer cultures compared to parent definitive endoderm cultures. The quantification was performed by staining cells with the mesoderm marker T and endoderm marker FOXA2 relative to the proliferative cell marker Ki67.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A method of producing one or more aggregated organoids, comprising:
differentiating definitive endoderm to a gut endoderm monolayer and gut spheroids, wherein the gut endoderm monolayer is adherent and the gut spheroids are detached and suspended in a growth medium;
separating the gut endoderm monolayer from the gut spheroids;
dissociating the gut endoderm monolayer to a single cell suspension of gut endoderm cells;
aggregating the single cell suspension of gut endoderm cells into one or more gut endoderm aggregates; and
culturing the one or more gut endoderm aggregates to produce the one or more aggregated organoids.

2. The method of claim 1, wherein the definitive endoderm has been differentiated from pluripotent stem cells.

3. The method of any one of the preceding claims, wherein the definitive endoderm has been differentiated from embryonic stem cells or induced pluripotent stem cells.

4. The method of any one of the preceding claims, wherein the definitive endoderm is human definitive endoderm.

5. The method of any one of the preceding claims, wherein the separating step comprises aspirating the growth medium and suspended gut spheroids from the gut endoderm monolayer.

6. The method of any one of the preceding claims, wherein the dissociating step comprises enzymatically dissociating the gut endoderm monolayer.

7. The method of claim 6, wherein the gut endoderm monolayer is enzymatically dissociated with Accutase, Accumax, trypsin, trypsin/EDTA, collagenase, dispase, TrypLE Express, or TrypLE Select, or any combination thereof.

8. The method of anyone of the preceding claims, wherein the aggregating step comprises aggregating the single cell suspension in hanging drops, centrifuging the single cell suspension in a “v” or “u”-bottomed microwell culture plate, aggregating the single cell suspension using an orbital shaker, or centrifuging the single cell suspension in a formation plate, or any combination thereof.

9. The method of claim 8, wherein the formation plate is an Aggrewell plate.

10. The method of any one of the preceding claims, wherein each of the one or more gut endoderm aggregates comprises about 250, about 500, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, or about 10000 gut endoderm cells, or any number of gut endoderm cells within a range defined by any two of the aforementioned number of cells.

11. The method of any one of the preceding claims, wherein the culturing step comprises contacting the one or more gut endoderm aggregates with an extracellular matrix, or mimetic or derivative thereof.

12. The method of claim 11, wherein the extracellular matrix, or mimetic or derivative thereof, comprises Matrigel.

13. The method of any one of claims 1-12, wherein the gut endoderm monolayer is a foregut endoderm monolayer and the gut spheroids are foregut spheroids.

14. The method of claim 13, wherein differentiating the definitive endoderm to the foregut endoderm monolayer and the foregut spheroids comprises contacting the definitive endoderm with one or more FGF signaling pathway activators, one or more Wnt signaling pathway activators, or one or more BMP signaling pathway inhibitors, or any combination thereof.

15. The method of claim 14, wherein the one or more FGF signaling pathway activators comprise FGF4, the one or more Wnt signaling pathway activators comprise CHIR99021, or the one or more BMP signaling pathway inhibitors comprise Noggin, or any combination thereof.

16. The method of any one of claims 13-15, wherein the one or more aggregated organoids are aggregated liver organoids.

17. The method of claim 16, wherein culturing the one or more gut endoderm aggregates to form the one or more aggregated liver organoids comprises contacting the one or more gut endoderm aggregates with one or more FGF signaling pathway activators, one or more BMP signaling pathway activators, retinoic acid, hepatocyte growth factor, dexamethasone, or Oncostatin M, or any combination thereof.

18. The method of claim 17, wherein the one or more FGF signaling pathway activators comprise FGF2, or the one or more BMP signaling pathway activators comprise BMP4, or both.

19. The method of any one of claims 13-15, wherein the one or more aggregated organoids are aggregated gastric organoids.

20. The method of claim 19, wherein the one or more aggregated gastric organoids are aggregated antral gastric organoids.

21. The method of claim 20, wherein culturing the one or more gut endoderm aggregates to form the one or more aggregated antral gastric organoids comprises contacting the one or more gut endoderm aggregates with EGF, retinoic acid, or one or more BMP signaling pathway inhibitors, or any combination thereof.

22. The method of claim 21, wherein the one or more BMP signaling pathway inhibitors comprise Noggin.

23. The method of any one of claims 1-12, wherein the gut endoderm monolayer is a hindgut endoderm monolayer and the gut spheroids are hindgut spheroids.

24. The method of claim 23, wherein differentiating the definitive endoderm to the hindgut endoderm monolayer and the hindgut spheroids comprises contacting the definitive endoderm with one or more FGF signaling pathway activators, or one or more Wnt signaling pathway activators, or both.

25. The method of claim 24, wherein the one or more FGF signaling pathway activators comprise FGF4, or the one or more Wnt signaling pathway activators comprise CHIR99021, or both.

26. The method of any one of claims 23-25, wherein the one or more aggregated organoids are aggregated intestinal organoids.

27. The method of claim 26, wherein culturing the one or more gut endoderm aggregates to form the one or more aggregated intestinal organoids comprises contacting the one or more gut endoderm aggregates with EGF, one or more Wnt signaling pathway activators, or one or more BMP signaling pathway inhibitors, or any combination thereof.

28. The method of claim 27, wherein the one or more Wnt signaling pathway activators comprise R-spondin, or the one or more BMP signaling pathway inhibitors comprise Noggin, or both.

29. The method of any one of claims 23-25, wherein the one or more aggregated organoids are aggregated colonic organoids.

30. The method of claim 29, wherein culturing the one or more gut endoderm aggregates to form the one or more aggregated colonic organoids comprises contacting the one or more gut endoderm aggregates with EGF, one or more Wnt signaling pathway activators, or one or more BMP signaling pathway activators, or any combination thereof.

31. The method of claim 30, wherein the one or more Wnt signaling pathway activators comprise R-spondin, or the one or more BMP signaling pathway activators comprise BMP2, or any combination thereof.

32. The method of any one of the preceding claims, wherein the one or more gut endoderm aggregates comprise at least 1, 10, 100, 200, 300, 400, 500, 600, 700, 800, 900, 1000, 2000, 3000, 4000, 5000, 6000, 7000, 8000, 9000, or 10000 gut endoderm aggregates, or any number of gut endoderm aggregates within a number defined by any two of the aforementioned number of gut endoderm aggregates.

33. The method of any one of the preceding claims, wherein each of the one or more gut endoderm aggregates comprise:
a diameter that is within ±10%, ±9%, ±8%, ±7%, ±6%, ±5%, ±4%, ±3%, ±2%, or 1% from the average diameter of the one or more gut endoderm aggregates, or any diameter within a range defined by any two of the aforementioned diameters; or
a volume that is within ±10%, ±9%, ±8%, ±7%, ±6%, ±5%, ±4%, ±3%, ±2%, or ±1% from the average volume of the one or more gut endoderm aggregates, or any volume within a range defined by any two of the aforementioned volumes; or both.

34. The method of any one of the preceding claims, further comprising transplanting the one or more aggregated organoids to a recipient subject.

35. The method of claim 34, wherein the recipient subject is a mammal.

36. The method of claim 34 or 35, wherein the recipient subject is a human.

37. The one or more aggregated organoids produced by any one of claims 1-36.

38. A plurality of gut endoderm aggregates, comprising at least 1000, 2000, 3000, 4000, 5000, 6000, 7000, 8000, 9000, or 10000 gut endoderm aggregates, or any number of gut endoderm aggregates within a number defined by any two of the aforementioned number of gut endoderm aggregates; wherein each of the plurality of gut endoderm aggregates comprises:
a diameter that is within ±10%, ±9%, ±8%, ±7%, ±6%, ±5%, ±4%, ±3%, ±2%, or ±1% from the average diameter of the plurality of gut endoderm aggregates, or any diameter within a range defined by any two of the aforementioned diameters; or
a volume that is within ±10%, ±9%, ±8%, ±7%, ±6%, ±5%, ±4%, ±3%, ±2%, or ±1% from the average volume of the plurality of gut endoderm aggregates, or any volume within a range defined by any two of the aforementioned volumes; or both.

39. The plurality of gut endoderm aggregates of claim 38, wherein the plurality of gut endoderm aggregates are derived from the same subject.

40. A formation plate comprising a plurality of microwells and the plurality of gut endoderm aggregates of claim 38 or 39, wherein each of the plurality of microwells comprises a single gut endoderm aggregate of the plurality of gut endoderm aggregates.

41. The plurality of gut endoderm aggregates of claim 38 or the formation plate of claim 40, wherein the plurality of gut endoderm aggregates is produced according to the methods of any one of claims 1-36.
